(19) United States
(12) Patent Application Publication
(10) Pub. No.: US 2025/0223552 A1
(43) Pub. Date: Jul. 10, 2025

(54) METHOD FOR PREPARING T CELLS FOR ADOPTIVE T CELL THERAPY

(71) Applicant: ST. JUDE CHILDREN'S RESEARCH HOSPITAL, INC., Memphis, TN (US)

(72) Inventors: Douglas R. GREEN, Memphis, TN (US); Hongbo CHI, Memphis, TN (US); Ao GUO, Memphis, TN (US); Hongling HUANG, Memphis, TN (US); Swantje LIEDMANN, Memphis, TN (US)

(21) Appl. No.: 18/847,889

(22) PCT Filed: Mar. 15, 2023
(86) PCT No.: PCT/US2023/064380

§ 371 Date: Sep. 17, 2024

Related U.S. Application Data
(60) Provisional application No. 63/321,339, filed on Mar. 18, 2022.

(51) Int. Cl. C12N 5/0783; A61K 40/11; A61K 40/31; A61P 35/00

(52) U.S. Cl. CPC C12N 5/0636; A61K 40/11; A61K 40/31; A61P 35/00; C12N 2501/2307; C12N 2501/2315; C12N 2501/51; C12N 2501/515; C12N 2510/00

(57) ABSTRACT

Disclosed is a method for preparing T cells for adoptive T cell therapy by contacting a population of activated T cells with an AT-rich interaction domain 1A (Aridla) inhibitor. Also disclosed is a kit, a population of T cells or engineered T cells produced by the method and use of the same in adoptive T cell therapy and the treatment of cancer.

### INTRODUCTION

This patent application claims the benefit of priority from U.S. Provisional Ser. No. 63/321,339, filed Mar. 18, 2022, the content of which is incorporated herein by reference in its entirety.

This invention was made with government support under grant nos. AI123322 and CA253188 awarded by the National Institutes of Health. The government has certain rights in this invention.

### BACKGROUND

Chimeric antigen receptor (CAR)-T cell therapy has changed the landscape of treatment options for B cell malignancies. However, frequent relapses in treated patients, together with inability to achieve complete remission in certain disease types, highlight the need of further potentiating this therapeutic strategy.

Extensive clinical experience has indicated that primary objective responses are associated with the level of CAR-T cell expansion early after infusion, while long-term persistence is required to prevent relapses. Among others, intrinsic T cell properties and composition of the infused T cell product have been reported to significantly shape CAR-T cell fitness. T cells exist in a wide range of interconnected differentiation statuses, differing in terms of proliferative capacity, self-renewal capabilities and long-term survival. In this regard, evidence in mice and humans suggests that T cell differentiation negatively correlates with long-term antitumor activity, with early memory T cells holding the most favorable features. Accordingly, T cells from chronic lymphocytic leukemia patients who responded to CD19 CAR-T cells were found enriched in gene expression profiles involved in early memory, or were rather the result of a single central memory T-cell (T_{CM}) clone deriving from a TET2-targeted insertional mutagenesis event (Majzner & Mackall (2019) Nat. Med. 25:1341-1355; Fraietta et al. (2018) Nat. Med. 24:563-571; Fraietta et al. (2018) Nature 558:307-312).

Therefore, there is a need in the art for generating T cells with memory function that persist and provide improved therapeutic benefits.

### SUMMARY OF THE INVENTION

This invention is based on the discovery that treatment of naïve T cells with an inhibitor of AT-rich interaction domain 1A (Aridla) during activation, and prior to adoptive T cell therapy promotes memory function of these cells and improves use of the same in, e.g., anti-cancer immunity. Accordingly, this invention provides a method for preparing T cells for adoptive T cell therapy by contacting a population of activated T cells, e.g., CD8⁺ T cells, with an Arid1a inhibitor, ideally during the first 48 hours after activation. In some aspects, activation of the population of T cells is via stimulation of CD3, CD28, or a combination thereof. In other aspects, the method further includes the step of expanding the activated population of T cells with one or more cytokines. In further aspects, the method includes the step of introducing into said population of T cells an exogenous nucleic acid molecule, e.g., an antigen recognizing receptor (e.g., a T cell receptor (TCR) or a chimeric antigen receptor (CAR)), an ortho-receptor, an immunomodulatory cytokine, a chemokine receptor, a dominant-negative receptor, or a transcription factor for preventing exhaustion, thereby producing a population of engineered T cells. Antigens of use in this aspect include, e. g., a tumor antigen, a self-antigen, or a pathogen antigen. Populations of T cells or engineered T cells prepared by the methods of this invention, and pharmaceutical compositions containing the same, are also provided, as are methods for using the T cells for adoptive T cell therapy and treating cancer. This invention further provides a kit for preparing T cells for adoptive T cell therapy, which includes (a) an Arid1a inhibitor, (b) CD3 agonist, and optionally (c) a costimulatory ligand and/or (d) one or more cytokines.

### BRIEF DESCRIPTION OF THE DRAWINGS

FIG. 1 shows quantification of the percentage of CD62L⁺CD44⁺ OT-I CD8+ T cell and the mean fluorescence intensity (MFI) of CD62L in OT-I CD8⁺ T cells treated with DMSO or Arid1a inhibitor (1 μM, BRD-K98645985) during activation with anti-CD3/CD28 for 48 hours followed additional 2 days culture with IL-2 (n=3 per group). Data are compiled from at least two independent experiments. Data are shown as mean±s.e.m. *p<0.05; two-tailed unpaired Student's t-test.

FIG. 2 shows tumor growth curve of B16-Ova tumors upon adoptive transfer of the indicated activated OT-I cells into tumor-bearing mice (n≥3 per group). Naïve OT-I CD8⁺ T cells were activated with anti-CD3/CD28 in the presence or absence of Arid1a inhibitor for 48 hours followed by expansion for another 4 days. Data are representative of two independent experiments. Data are as shown mean±s.e.m. **p<0.01, ***p<0.001; two-way ANOVA.

FIG. 3 shows tumor growth curve of MC38-Ova tumors upon adoptive transfer of the indicated activated OT-I cells into tumor-bearing mice (n≥3 per group). Naïve OT-I CD8⁺ T cells were activated with anti-CD3/CD28 in the presence or absence of Arid1a inhibitor for 48 hours followed by expansion for another 4 days. Data are representative of two independent experiments. Data are shown as mean±s.e.m. **p<0.01; two-way ANOVA.

FIG. 4 shows expansion of B7-H3 CAR-T cells pretreated with or without Arid1a inhibitor during repeat stimulation with B7-H3-expressing GL261 tumor cells in vitro (n=3 per group). T cells were transduced with B7-H3 CAR or a control CAR containing only the single-chain variable fragment (scFv) and transmembrane domain (B7-H3 STOP CAR) and co-cultured with GL261 tumor cells at the ratio of 3:1. Cells numbers were assessed every 3 days. Data are compiled from at least two independent experiments. Data are shown as mean±s.e.m. ***p<0.001; two-way ANOVA.

FIG. 5 shows expansion of B7-H3 CAR-T cells pretreated with or without Arid1a inhibitor during repeat stimulation with B7-H3-expressing F420 tumor cells in vitro (n=3 per group). T cells were transduced with B7-H3 CAR or a control CAR containing only the scFv and transmembrane domain (B7-H3 STOP CAR) and co-cultured with F420 tumor cells at the ratio of 3:1. Cells numbers were assessed every 3 days. Data are compiled from at least two independent experiments. Data are shown as mean±s.e.m. *p<0.05; two-way ANOVA.

FIG. 6 shows a tumor growth curve of F420 tumor-bearing mice treated with B7-H3 CAR-T cells pre-treated with or without Arid1a inhibitor during T cell activation (n=6 per group). Data are representative of two independent experiments. Data are shown as mean±s.e.m. *p<0.05, ***p<0.001; two-way ANOVA.

FIG. 7 shows percent survival of F420 tumor-bearing mice treated with B7-H3 CAR-T cells pre-treated with or without Arid1a inhibitor during T cell activation (n=6 per group). Data are representative of two independent experiments. Log-rank (Mantel-Cox) test was performed for comparing survival curves.

FIG. 8 shows quantification of the proportions of human T_{CM} (CD45RA⁻CCR7⁺CD8⁺), T_{EM} (CD45RA⁻CCR7⁻CD8⁺), and T_{SCM} (CD45RA⁺CCR7⁺CD27⁺CD95⁺) cells in vitro. Naïve human CD8⁺ T cells were activated with anti-CD3/CD28 and cultured with IL-15; Arid1a inhibitor was added at the indicated time point (n=6 per group). Data are compiled from at least two independent experiments. Data are representative of two independent experiments. Data shown are as mean±s.e.m. *p<0.05, **p<0.01, ***p<0.001; one-way ANOVA.

FIG. 9 shows the quantification of donor-derived total, T_{CM}-like (CD45RA⁻CD62L⁺) and T_{EM}-like (CD45RA⁻CD62L⁻) human CD8⁺ T cells in Nod-Scid-common gamma chain-deficient (NSG) mice at day 30 after adoptive transfer of human CD8⁺ T cells. Naïve human CD8⁺ T cells were activated with anti-CD3/CD28 in the presence or absence of Arid1a inhibitor for 48 hours followed by expansion in IL-15 containing medium for another 4 days prior to adoptive transfer. Data are representative of two independent experiments. Data are shown as mean±s.e.m. *p<0.05; two-tailed unpaired Student's t-test.

### DETAILED DESCRIPTION OF THE INVENTION

It has now been found that transient treatment of activated T cells with an Arid1a inhibitor promotes memory function and enhanced efficacy compared to conventionally activated T cells. Notably, transient inhibition of Arid1a during preparation of CAR-T cells, in particular at approximately the time of the first division, results in control of tumor growth and improved anti-tumor immunity.

According, the present invention provides a method for preparing T cells for adoptive T cell therapy by contacting a population of activated T cells with an Arid1a inhibitor. The terms “T cell” or “T lymphocyte” are art-recognized and are intended to include thymocytes, naïve T lymphocytes, immature T lymphocytes, mature T lymphocytes, resting T lymphocytes, or activated T lymphocytes.

Illustrative populations of T cells suitable for use in the methods of this invention include but are not limited to helper T cells (HTL; CD4⁺ T cells), cytotoxic T cells (CTL; CD8⁺ T cells), CD4⁺CD8⁺ T cells, or any other suitable subset of T cells. Other illustrative populations of T cells suitable for use include T cells expressing one or more of the following markers: CD3, CD4, CD8, CD27, CD28, CD45RA, CD45RO, CD62L, CD127, CD197, and HLA-DR. In particular aspects, the population of T cells comprises, consists essentially of, consists of, or is composed substantially of (e.g., more than 90%, 95%, 97%, 98%, 99%) CD8⁺ T cells.

T cells for adoptive T cell therapy may be autologous/autogeneic (“self”) or non-autologous (“non-self,” e. g., allogeneic, syngeneic, or xenogeneic). “Autologous” refers to cells from the same subject. “Allogeneic” refers to cells of the same species that differ genetically to the cell in comparison. “Syngeneic” refers to cells of a different subject that are genetically identical to the cell in comparison. “Xenogeneic” refers to cells of a different species to the cell in comparison.

In one aspect, the T cells are obtained from a mammalian subject. In another aspect, the cells are obtained from a primate subject. In a particular aspect, the cells are obtained from a human subject. The population of T cells can be obtained from a number of sources including, but not limited to, peripheral blood, peripheral blood mononuclear cells, bone marrow, lymph nodes tissue, cord blood, thymus issue, tissue from a site of infection, ascites, pleural effusion, spleen tissue, and tumors. In certain aspects, the population of T cells are isolated from the circulating blood of an individual by apheresis, e.g., leukapheresis. The apheresis product may contain lymphocytes, including T cells, monocytes, granulocytes, B cells, other nucleated white blood cells, red blood cells, and platelets or may be a leukapheresis product including lymphocytes, including T cells, monocytes, granulocytes, B cells, and other nucleated white blood cells. The cells collected by apheresis may be washed to remove the plasma fraction and to place the cells in an appropriate buffer or media for subsequent processing. The cells can be washed with phosphate-buffered saline (PBS) or with another suitable solution that lacks calcium, magnesium, and most, if not all other, divalent cations.

Once a population of cells containing T cells has been obtained, cell counts and viability of cells within the population of cells can be determined, the population or portions thereof may be cryopreserved for future use or analyses, and cells in the population, e.g., PBMCs, may be characterized using a number of cell marker panels, e.g., CD3, CD4, CD8, CD14, CD16, CD19, CD28, CD45RA, CD45RO, CD61, CD62L, CD66b, CD127, and HLA-DR, and maintained in T cell culture medium.

In particular aspects, a population of PBMCs is used to isolate a population of T cells. Specific cell types can be isolated from PBMCs as described herein or by conventional methods. In some aspects, cytotoxic and helper T lymphocytes can be sorted into naïve, memory, and effector T cell subpopulations either before or after activation, expansion, and/or genetic modification. As an alternative, T cells may be obtained commercially, e.g., Sanguine Biosciences.

Once isolated, the population of T cells is activated. The terms “activated” or “activation” refer to the state of a T cell that has been sufficiently stimulated to induce detectable cellular proliferation. In some aspects, activation can also be associated with induced cytokine production. The term “activated T cells” refers to, among other things, T cells that are proliferating. As is conventional in the art, “stimulation” refers to a primary response induced by binding of a stimulatory molecule with its cognate ligand thereby mediating a signal transduction event including, but not limited to, signal transduction via the TCR/CD3 complex or via stimulation of the CD2 surface protein. Preferably, the population of T cells of this invention are activated by stimulating CD3. In some aspects, stimulation of CD3 is carried out with a CD3 agonist. A suitable CD3 agonist includes aa CD3 ligand or anti-CD3 antibody, in particular an activating antibody. Illustrative examples of CD3 antibodies include, but are not limited to, OKT3, G19-4, BC3, and 64.1.

Signals generated through the TCR alone are often insufficient for full activation of T cells and one or more secondary or costimulatory signals may be required. Thus, T cell activation may include the use of a primary stimulation signal through the TCR/CD3 complex and one or more secondary costimulatory signals. A costimulatory signal can be achieved using a costimulatory ligand including, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor or a ligand that specifically binds with B7-H3. A costimulatory ligand also encompasses, inter alia, an antibody or antigen binding fragment thereof that specifically binds with a costimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83. In certain aspects, the T cell is activated by costimulation of CD28, e.g., with a CD28 ligand or anti-CD28 antibody. Illustrative examples of suitable anti-CD28 antibodies include monoclonal antibodies 9.3, B-T3, XR-CD28, KOLT-2, 15E8, 248.23.2, and EX5.3D10. The stimulation of CD3 and optionally a costimulatory molecule such as CD28 may be performed according to any known method in the art for instance beads, matrix, or cell-free matrix. Alternatively, aAPCs expressing anti-CD3 and anti-CD28 single chain variable fragments (scFvs) may be used (Shrestha et al. (2020) J. Immunother. 43 (3): 79-88).

To promote memory function of a population of activated T cells, this invention provides for contacting the population of activated T cells with an Arid1a inhibitor. As is known in the art, Arid1a (BAF250A) is a component of the mammalian SWI/SNF (or BAF, for BRG1/BRM-associated factor) complex. The BAF complex is an ATP-dependent chromatin remodeler composed of 12-15 subunits that regulates genomic architecture and DNA accessibility. In addition to Aridla, the BAF complex may include, e.g., SMARCA4 (BRG1), SMARCA2 (BRM), ARID1B (BAF250B), BCL11A, BCL11B, BCL7A, BCL7B, BCL7C, SMARCB1 (BAF47), SMARCD1 (BAF60A), SMARCD2 (BAF60B), SMARCD3 (BAF60c), SMARCC1 (BAF155), SMARCC2 (BAF170), DPF1 (BAF45B), DPF2 (BAF45C), DPF3 (BAF45D), ACTL6A (BAF53A), ACTL 6B (BAF53B), BRD9, BRD7, SS18, CREST (SS18L1), and SMARCE1 (BAF57).

An Arid1a inhibitor may be any suitable molecule that can transiently inhibit the expression or activity of Aridla or Aridla-specific BAF complexes. Such molecules can include, but are not limited to, small molecule inhibitors, as well as protein/peptide-based inhibitors or inhibitory RNA (iRNA) molecules (e.g., siRNA or shRNA) that are transiently expressed to disrupt Arid1a or Aridla-specific BAF complex activity or expression. Exemplary iRNA constructs targeting Arid1A include, e.g., siGenome L-017263-00-0005 (Dharmacon) and TRCN0000059091: shRNA-1 and TRCN0000059090: shRNA-2 (Raab et al. (2015) PLOS Genet. 11: e1005748). Small molecule inhibitors of Arid1A or Aridla-specific BAF complex activity include Baficillin 1 or RBD-K98645985 (MedChemExpress or AOBIOUS) (CAS No. 1357647-78-9) or analogs thereof including, but not limited to BRD-K51299478, BRD-K80443127, BRD-K49078264, BRD-K55993513, BRD-K25923209, BRD-K98645985, BRD-K17257309, and BRD-K13449002. See, also, US 2020/255416A1 and Marian et al. (2018) Cell Chem. Biol. 25:1443-1455.

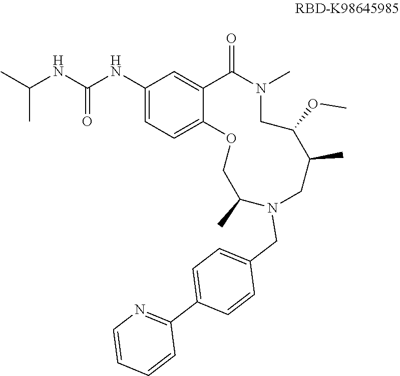 [SMILES: CO[C@H]1CN(C)C(=O)c2cc(NC(=O)NC(C)C)ccc2OC[C@H](C)N(Cc2ccc(-c3ccccn3)cc2)C[C@@H]1C]

Other BAF complex inhibitors may also be used. Illustrative examples of such inhibitors include, but are not limited to, phospho-aminoglycosides (phospho-kanamycin, aka ADAADi); which inhibit yeast the SWI2/SNF2 complex (Muthuswami et al. (2000) Biochemistry 39 (15): 4358-65); dual BRG1 and BRM inhibitor (Papillon et al. (2018) J. Med. Chem. 61 (22): 10155-10172); and BAF inhibitors A01, A11, and C09 (Dykhuizen et al. (2012) J. Biomol. Screen. 17:1221-1230; Stoszko et al. (2016) EBioMedicine 3:108-121).

As demonstrated herein, transient contact of population of T cells, in particular CD8⁺ T cells, promoted memory cell phenotypes. As used herein, “transient” refers to lasting only for a brief time, e.g., less than 8 to 10 days. Notably, treatment during the first 48 hours promoted the generation of cells with markers of T_{CM} (CD45RA-CCR7+) and T_{SCM} (CD45RA⁺CCR7⁺CD27⁺CD95) and reduced numbers of cells with markers of T_{EM} (CD45RA⁺CCR7⁻). Accordingly, in certain aspects of this invention, the population of activated T cells, e.g., CD8⁺ T cells or CD4⁺ T cells, is contacted with the Aridla inhibitor immediately after activation and up to 48 hours, 72 hours, 96 hours, 102 hours, 168 hours, or 192 hours after activation. In particular aspects, the population of activated T cells is contacted with the Arid1a inhibitor immediately during the first 48 hours after activation, i.e., during the period between 0 hours and 48 hours after activation. Subsequently, the Arid1a inhibitor is removed, e.g., by washing to cells.

In some aspects of the invention, the method of the invention further includes expanding the activated population of T cells in one or more cytokines. Ideally, the T cells are expanded during and/or after activation and/or during and/or after contact of the T cells with the Arid1a inhibitor. Exemplary cytokines of use in expanding the activated population of T cells include, but are not limited to, IL-2, IL-15, IL-7, IL-9, IL-21, IL-23, or a combination thereof.

In certain aspects, the method further includes the step of introducing in the T cells of said population of T cells an exogenous nucleic acid molecule thereby producing an engineered T cell. As used herein, the term “engineered T cell,” “genetically engineered T cell” or “genetically modified T cell” refers to the addition of extra genetic material in the form of DNA or RNA into the total genetic material of the T cell. Preferably said introduction is performed before the expansion of the cells. Preferably the exogenous nucleic acid molecule encodes an antigen-recognizing receptor, an ortho-receptor, an immunomodulatory cytokine, a chemokine receptor, a dominant-negative receptor (for instance PD1 DDR; see Cherkassky et al. (2016) J. Clin. Invest. 126 (8): 3130-44), a transcription factor for preventing exhaustion (such as c-june; see Lynn et al. (2019) Nature 576 (7786): 293-300). In certain aspects, the antigen is a tumor antigen, a self-antigen, or a pathogen antigen. Preferably said antigen recognizing receptor is a T cell receptor (TCR) or a chimeric antigen receptor (CAR).

A TCR is a molecule which can be found on the surface of T cells that is responsible for recognizing antigens bound to MHC molecules. The naturally occurring TCR heterodimer is composed of an alpha (α) and beta (β) chain in approximately 95% of T cells, whereas about 5% of T cells have TCRs composed of gamma (γ) and delta (δ) chains. Engagement of a TCR with antigen and MHC results in activation of the T lymphocyte on which the TCR is expressed. Each chain of a natural TCR is a member of the immunoglobulin superfamily and possesses one N-terminal immunoglobulin Ig-variable (V) domain, one Ig-constant (C) domain, a transmembrane/cell membrane-spanning region, and a short cytoplasmic tail at the C-terminal end. The variable domain of both the TCR α chain or β chain have three hypervariable or complementarity determining regions (CDRs). A constant domain of a TCR may be composed of short connecting sequences in which a cysteine residue forms a disulfide bond, making a link between the two chains. An α chain of a TCR of the present invention may have a constant domain encoded by a TRAC gene. A β chain of a TCR of the present invention may have a constant domain encoded by a TRBC1 or a TRBC2 gene.

A CAR is an engineered receptor, which can confer an antigen specificity onto cells (for example T cells). CARS are also known as artificial T cell receptors, chimeric T cell receptors or chimeric immunoreceptors. Preferably the CARs of the invention include an antigen-specific targeting region, an extracellular domain, a transmembrane domain, optionally one or more co-stimulatory domains, and an domain. The antigen-specific intracellular signaling targeting domain provides the CAR with the ability to bind to the target antigen of interest. The antigen-specific targeting domain preferably targets an antigen of clinical interest against which it would be desirable to trigger an effector immune response that results in tumor killing. The antigen-specific targeting domain may be any protein or peptide that possesses the ability to specifically recognize and bind to a biological molecule (e.g., a cell surface receptor or tumor protein, or a component thereof). The antigen-specific targeting domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins.

Examples of antigens which may be targeted by the CAR of the invention include but are not limited to antigens expressed on cancer cells and antigens expressed on cells associated with various hematologic diseases, autoimmune diseases, inflammatory diseases, and infectious diseases. With respect to targeting domains that target cancer antigens, the selection of the targeting domain will depend on the type of cancer to be treated. Examples of antigens specific for cancer, which may be targeted by a CAR, include but are not limited to any one or more of mesothelin, EGERvIII, TSHR, CD19, CD123, CD22, CD30, CD171, CS-1, CLL-1, CD33, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGER2, Lewis-Y, CD24, platelet-derived growth factor receptor-beta (PDGFR-beta), SSEA-4, CD20, Folate receptor α (FRα), ERBB2 (Her2/neu), MUC1, epidermal growth factor receptor (EGFR), NCAM, Prostase, PAP, ELF2M, Ephrin B2, IGF-I receptor, CAIX, LMP2, gp100, bor-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. Preferably, the antigen-specific binding domain specifically binds to a tumor antigen.

Antigens specific for inflammatory diseases which may be targeted by the CAR of the invention include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), MEDI-528, myostatin, OX-40, rhuMAbb7, scleroscin, SOST, TGF-β, TNF-α or VEGF-A.

Antigens specific for neuronal disorders which may be targeted by the CAR of the invention include but are not limited to any one or more of beta amyloid or MABT5102A.

Antigens specific for cardiovascular diseases which may be targeted by the CARs of the invention include but are not limited to any one or more of C5, cardiac myosin, CD41 (integrin alpha-IIb), fibrin II, beta chain, ITGB2 (CD18) and sphingosine-1-phosphate.

The CAR also includes one or more co-stimulatory domains. This domain may enhance cell proliferation, cell survival and development of memory cells. Each co-stimulatory domain includes the co-stimulatory domain of any one or more of, for example, a MHC class I molecule, a TNF receptor protein, an immunoglobulin-like protein, a cytokine receptor, an integrin, a signaling lymphocytic activation molecule (SLAM protein), an activating NK cell receptor, BTLA, a Toll ligand receptor, 0X40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8α, CD83, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (CD150, IPO-3), BLAME (SLAMF8), SELPLG (CD162), LTBR, LAT, GADS, SLP-76, PAG/Cbp, CD19a, and a ligand that specifically binds with CD83. Additional co-stimulatory domains will be apparent to those of skill in the art.

The CAR also includes an intracellular signaling domain. This domain may be cytoplasmic and may transduce the effector function signal and direct the cell to perform its specialized function. Examples of intracellular signaling domains include, but are not limited to, ζ chain of the T-cell receptor or any of its homologs (e.g., η chain, FcεRIγ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may be human CD3ζ chain, FcγRIII, FcεRI, cytoplasmic tails of Fc receptors, immunoreceptor tyrosine-based activation motif (ITAM) bearing cytoplasmic receptors or combinations thereof.

The CAR also includes a transmembrane domain. The transmembrane domain may be the transmembrane sequence from any protein which has a transmembrane domain, including any of the type I, type II, or type III transmembrane proteins. The transmembrane domain of the CAR of the invention may also be an artificial hydrophobic sequence. The transmembrane domains of the CARs of the invention may be selected so as not to dimerize. Examples of transmembrane (TM) regions used in CAR constructs may be obtained from CD28, OX40, 4-1BB, CD3ζ, or CD8a. Additional transmembrane domains will be apparent to those of skill in the art.

An exogenous nucleic acid molecule may be introduced into T cells of the invention by a vector such as an adenovirus, retrovirus, or lentivirus-based vector, or endonucleases, such as CRISPR-associated (CRISPR/Cas9, Cpf1, and the like) nucleases. Other suitable delivery systems include, e.g., DNA transfection methods such as electroporation, DNA biolistics, lipid-mediated transfection, compacted DNA-mediated transfection, liposomes, immunoliposomes, cationic agent-mediated transfection, cationic facial amphiphiles (CFAs) and combinations thereof. Preferably said exogenous nucleic acid molecule is placed at an endogenous gene locus of the T cell. Preferably said introduction of the said exogenous nucleic acid molecule disrupts or abolishes expression of an endogenous TCR.

In an alternative aspect, T cells of the invention are specific for an antigen, e.g., a pathogen or tumor antigen as described herein, by culturing the cells in the presence of an antigen. By way of illustration, tumor antigen-specific CD8⁺ T cells may be generated by culturing lymphocytes from PBMCs in the presence of a tumor antigen, an antigen presenting cell such as a dendritic cell, IL-21, IL-15, and rapamycin and preferably in the absence of IL-2.

The invention also provides population of T cells or engineered T cells produced or obtainable by the methods of the invention. Preferably said produced or obtained T cells or engineered T cells are isolated (i.e., at least 90%, 95%, 978, 98%, 99% or 99.9% homogenous to said T cells). The invention also provides a population of CAR T cells obtainable by the method of the invention or a population of TCR-engineered T cells obtainable by the method of the invention. Ideally, the population of T cells or engineered T cells of this invention are composed of about 40% to 45% CD45RA⁻CCR7⁺ central memory T and cells about 8% to 12% CD45RA⁺CCR7⁺CD27⁺CD95⁺ stem cell memory T cells.

For therapeutic applications, it is preferable that the population of T cells or engineered T cells described herein are prepared in the form of a pharmaceutical composition including said T cells in admixture with a pharmaceutically acceptable carrier or vehicle. A “pharmaceutical composition” refers to a composition formulated in pharmaceutically acceptable or physiologically acceptable solutions for administration to a cell or an animal, either alone, or in combination with one or more other modalities of therapy. It will also be understood that, if desired, the compositions of the invention may be administered in combination with other agents as well, such as, e.g., cytokines, growth factors, hormones, small molecules, chemotherapeutics, pro-drugs, drugs, antibodies, or other various pharmaceutically active agents. The phrase “pharmaceutically acceptable” is used herein to refer to those compounds, materials, compositions, and/or dosage forms which are, the scope of sound medical judgment, suitable for use in contact with the tissues of human beings and animals without excessive toxicity, irritation, allergic response, or other problem or complication, commensurate with a reasonable benefit/risk ratio.

Suitable pharmaceutically acceptable carriers or vehicles of use in this invention include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular aspects, compositions of the present invention include an effective amount of the T cells prepared by the methods described herein. It can generally be stated that a pharmaceutical composition including the T cells prepared by the methods of this invention may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-7, IL-15, IL-12, TNF-α, IL-18, TNF-β, GM-CSF, IL-4, IL-13, Flt3-L, RATES, MIPIα, etc.) to enhance engraftment and function of infused T cells.

Pharmaceutical compositions of the present invention are preferably formulated for parenteral administration, e.g., intravascular (intravenous or intraarterial), intraperitoneal or intramuscular administration. In one aspect, the pharmaceutical compositions are administered intravenously.

In some aspects, pharmaceutical compositions of the invention include an effective amount of an expanded population of T cells, alone or in combination with one or more therapeutic agents. Thus, the T cells may be administered alone or in combination with other known cancer treatments, such as radiation therapy, chemotherapy, transplantation, immunotherapy, hormone therapy, photodynamic therapy, etc. The T cells may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDS, DMARDS, anti-inflammatories, chemotherapeutics, radiotherapeutics, therapeutic antibodies, or other active and ancillary agents.

Generally, compositions including the cells activated and expanded as described herein may be used in a subject (e.g., a mammal such as a human or primate) in need of adoptive T cell therapy. “Adoptive T cell therapy” (or adoptive cell transfer or cellular adoptive immunotherapy or T-cell transfer therapy) refers to a type of immunotherapy in which T cells are given to a subject to help the body fight diseases. Typical subjects include humans that have a cancer, infectious disease, immunodeficiency, inflammatory disease, or auto-immune disorder, which have been diagnosed with a cancer, infectious disease, immunodeficiency, inflammatory disease, or auto-immune disorder, or that are at risk or having a cancer, infectious disease, immunodeficiency, inflammatory disease, or auto-immune disorder. Use of the cells prepared in accordance with the methods described herein increase persistence and better response to the T cells in subjects treated with the same as compared to subjects treated with conventional T cells (i.e., T cells not contacted with an Arid1a inhibitor).

In certain aspects, compositions including the T cells prepared by the methods described herein are used in the conditions treatment of various including, without limitation, cancer, infectious disease, autoimmune disease, inflammatory disease, and immunodeficiency. As used herein, “treatment” or “treating” includes any beneficial or desirable effect on the symptoms or pathology of a disease or pathological condition and may include even minimal reductions in one or more measurable markers of the disease or condition being treated, e.g., cancer. Treatment can involve optionally either amelioration of, or complete reduction of, one or more symptoms of the disease or condition, or the delaying of the progression of the disease or condition. “Treatment” does not necessarily indicate complete eradication or cure of the disease or condition, or associated symptoms thereof.

In particular, the cells of this invention are of use in the treatment of solid tumors or cancers including, without limitation, liver cancer, bone cancer, pancreatic cancer, lung cancer, breast cancer, bladder cancer, brain cancer, bone cancer, thyroid cancer, kidney cancer, ovarian cancer, colon cancer, testicular cancer, head and neck cancer, stomach cancer, cervical cancer, rectal cancer, esophageal cancer, uterine cancer, prostate cancer or skin cancer. Moreover, the cells of the invention are of use in the treatment of leukemia, including acute leukemia (e.g., acute lymphocytic leukemia (ALL), acute myelocytic leukemia (AML), and myeloblasts, promyelocytic, myelomonocytic, monocytic and erythroleukemia), chronic leukemia (e.g., chronic lymphocytic leukemia (CLL), small lymphocytic lymphoma (SLL), chronic myelogenous leukemia (CML), Hairy cell leukemia (HCL)), polycythemia vera, lymphoma, Hodgkin's disease, non-Hodgkin's lymphoma, multiple myeloma, Waldenstrom's macroglobulinemia, and heavy chain disease.

In addition to cancer, a variety of diseases or conditions may be ameliorated by introducing the T cells of the invention to a subject in need of adoptive T cell therapy. Examples of diseases including various autoimmune disorders such as alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopeniaurpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus, erythematosus, some forms of thyroiditis, some forms of uveitis, vitiligo, granulomatosis with poly angiitis (Wegener's); and infections, including but not limited to, HIV (human immunodeficiency virus), RSV (Respiratory Syncytial Virus), EBV (Epstein-Barr virus), CMV (cytomegalovirus), HBV (hepatitis B virus), HCV (hepatitis C virus), adenovirus, coronavirus (e.g., SARS-CoV2) and BK polyomavirus infections.

To facilitate the preparation of T cells for adoptive T cell therapy, the invention also provides a kit including (a) an Arid1a inhibitor, and (b) a CD3 agonist, as described herein. In some aspects, the kit further includes one or more costimulatory ligands, e.g., an anti-CD28 antibody, and optionally one or more cytokines, e.g., IL-2, IL-15, IL-7, IL-23, or a combination thereof. Kits typically include a label indicating the intended use of the contents of the kit and instructions for use. The term “label” includes any writing, or recorded material supplied on or with the kit, or which otherwise accompanies the kit. In some aspects, the instructions provide the steps used in preparing T cells for adoptive T cell therapy including obtaining a suitable population of T cells (e.g., autologous/autogeneic CD8+ T cells isolated from PBMCs of a subject diagnosed with cancer); contacting the T cells with the CD3 agonist (e.g., an anti-CD3 antibody) optionally in the presence of a costimulatory ligand (e.g., an anti-CD28 antibody) for a time sufficient to activate the T cells; and contacting the activated T cells with an effective amount of an Arid1a inhibitor to produce T cells with memory function. The instructions can further include steps for expanding the T cells during and/or after activation with one or more cytokines (e.g., IL-2 or IL-15); introducing exogenous nucleic acid molecules (e.g., a nucleic acid molecule encoding a CAR); and administering the cells to a subject in need of treatment. The kit can also optionally include culture vessels, culture medium, wash solutions, and the like.

The following non-limiting examples are provided to further illustrate the present invention.

### Example 1: Materials and Methods

Mice. Mice, including both sexes, were used for the study. Rosa26-Cas9 knock-in mice (Platt et al. (2014) Cell 159:440-455) were crossed with OT-I (Hogquist et al. (1994) Cell 76:17-27) transgenic mice to express Cas9 in antigen-specific CD8⁺ T cells (referred to as Cas9-OT-I 592 mice). The Cas9 mice were fully backcrossed to the C57BL/6 background. Arid1a^{fl/fl} mice (Mathur et al. (2017) Nat. Genet. 49:296-302) and c-Myc-GFP fusion knock-in mice (Huang et al. (2008) Eur. J. Immunol. 38:342-349) have been previously described. T cell-specific deletion of Pbrm1 (Wurster et al. (2012) BMC Immunol. 13:9) was achieved by breeding Cd4^{Cre} mice with Pbrm1^{fl/fl} mice. c-Myc^{fl/fl} mice were obtained from F. Alt (de Alboran et al. (2001) Immunity 14:45-55) and were backcrossed five generations onto the C57BL/6 background before breeding to Rosa26^{Cre-ERT2} mice (Badea et al. (2003) J. Neurosci. 23:2314-2322). C57BL/6 and Rag1^{-/-} mice were purchased from the Jackson Laboratory. All mice were housed in specific-pathogen-free conditions in the Animal Resource Center at St Jude Children's Research Hospital. Mouse studies were conducted in accordance with protocols approved by the St. Jude Children's Research Hospital Committee on Care and Use of Animals and in compliance with all relevant ethical guidelines. All animal experiments were performed with age-matched littermate controls.

Human Naïve CD8⁺ T Cell Isolation and Stimulation. Human naive CD8⁺ T cells were isolated from apheresis ring obtained from the St. Jude blood donor center. Peripheral blood mononuclear cells (PBMC) were isolated using a density gradient medium sold under the tradename LYMPHOPREP® (StemCell Inc.) following the manufacturer's protocol. Briefly, blood from apheresis ring was mixed with phosphate-buffered saline (PBS) containing 2% fetal bovine serum in 1:1 ratio. The mixture was then layered carefully on a density gradient medium sold under the tradename LYMPHOPREP®. Samples were centrifuged at 4000 rpm for 30 minutes at room temperature without braking. The PBMC layer was aspirated, washed twice with 2% fetal calf serum/PBS before isolation of naïve CD8⁺ T cell using the MojoSort Human CD8⁺ Naïve T Cell Isolation Kit (Biolegend). Naïve CD8⁺ T cells were then stimulated with human T cell activation and expansion reagent sold under the tradename IMMUNOCULT® Human CD3/CD28 T Cell Activator (StemCell Inc.) plus 50 U/ml rhIL-2 or 10 ng/ml rhIL-15 for 2 days and expanded with 50 U/ml rhIL-2 or 10 ng/ml rhIL-15. BRD-K98645985 (1 μM; AOBIOUS or MedChemExpress) was added at the indicated time points. All human studies were in compliance with the Declaration of Helsinki. Blood donors were recruited by the Blood Donor Center at St. Jude Children's Research Hospital. Blood donors provided written consent for research use of their blood products not used in transfusions, which has been reviewed and approved by the Institutional Review Board at St. Jude Children's Research Hospital.

Viral Transduction and Genome Editing Efficiency Measurement. Naïve Cas9-expressing OT-I cells were isolated from the spleen and peripheral lymph nodes (pLN) of Cas9-OT-I mice by magnetic bead purification using a naïve CD8α⁺ T cell isolation kit according to the manufacturer's instructions (Miltenyi Biotech). Purified naïve ovalbumin (Ova)-specific CD8⁺ T (OT-I) cells were activated in vitro for 18 hours with plate-bound anti-CD3ε (10 μg/ml; Bio-X-Cell) and anti-CD28 (5 μg/ml; Bio-X-Cell) antibodies. Viral transduction was performed by spin-infection at 900 g at 25° C. for 3 hours with 10 μg/ml polybrene (Sigma-Aldrich) followed by resting for 3 hours at 37° C. and 5% CO₂. Cells were washed and cultured in media supplemented with murine IL-7 (12.5 ng/ml; PeproTech) and IL-15 (25 ng/ml; PeproTech) for 4 days. Cells were sorted based on the expression of fluorescent proteins using a Reflection cell sorter (iCyt) or MoFlo XDP cell sorter before adoptive transfer to recipients. At least two independent guides were designed using the platform from Broad Institute for phenotypic analyses. It was verified that at least two guides induced similar phenotypes. The sgRNAs editing efficiency was measured by insertion and deletion (indel) mutation analysis using CRIS.py (Connelly & Pruett-Miller (2019) Sci. Rep. 9:4194).

Adoptive Transfer, In Vivo Infection and Recall Assays. For influenza A virus infection, 5×10⁵ naïve or first-division c-Myc-GFP^{hi} and c-Myc-GEP^{lo} OT-I CD8⁺ T cells were transferred into CD45.1⁺ recipient animals intravenously (i.v.), which were infected with 4×10³ median embryo infectious dose (EID₅₀) influenza A virus A/X-31 (H3N2) expressing ovalbumin peptide (SIINFEKL; SEQ ID NO: 1). Spleens were collected and analyzed by flow cytometry at nine days after the infection. For Listeria monocytogenes infection, naïve OT-I cells (5×10³) or retrovirus-transduced (1-2×10⁴) OT-I cells were adoptively transferred i.v. into naïve C57BL/6 mice (for analysis at effector phase) or into Cas9-expressing hosts (for analysis at memory phase). To examine cell-intrinsic effects, the dual-color transfer systems were applied. Specifically, cells transduced with the indicated sgRNAs (marked by the expression of Ametrine) were mixed at a 1:1 ratio with those transduced with sgNTC (labelled with GFP), followed by adoptive transfer to the same host. For pathogen-induced infections, 3×10⁴ clone-forming units (CFU) of Listeria monocytogenes expressing ovalbumin (Lm-Ova) were injected i.v. To evaluate T_{MEM} recall responses, 5×10³ splenic T_{MEM} cells were sorted and transferred to naïve C57BL/6 hosts and re-challenged with 5×10⁴ CFU of Lm-Ova one day after T_{MEM} transfer. The recall responses were analyzed at day 6 after re-challenge. To examine the homeostatic proliferation, a total of 5×10⁵ T_{MEM} cells were labeled with a fluorescent dye sold under the tradename CELLTRACE® Violet (ThermoFisher Scientific) and transferred into Rag1^{-/-} mice and then analyzed at day 7 post-transfer.

For human CD8⁺ T cell experiments, naïve CD8⁺ T cells were activated by human CD3/CD28 T cell activator (StemCell) with or without an Arid1a inhibitor (BRD-K98645985; 1 μM; AOBIOUS or MedChemExpress) for two days and cultured in rhIL-15 (10 ng/ml, Peprotech) containing medium. Activated CD8⁺ T cells (3×10⁶) were then transferred to NOD scid gamma mice sold under the tradename NSG®. After 30 days, splenic cells were analyzed by flow cytometry.

CRISPR-Cas9 Mutagenesis Screening Using the Retroviral Epigenetic Library: Retroviral SgRNA Epigenetic Library Construction. The retroviral sgRNA vector used for library construction was previously described (Huang et al. (2021) Cell 184:1245-1261 e1221). A custom mouse epigenetic library targeting 337 genes were selected and guide RNA sequences were designed according to previously published criteria (Sanson et al. (2018) Nat. Commun. 9:5416). The library contains a total of 2,222 gRNAs with six gRNAs targeting one gene and 200 non-targeting controls. The synthesis, purification, and quality control of the library were according to established methods (Wei et al. (2019) Nature 576:471-476).

CRISPR-Cas9 Mutagenesis Screening Using the Retroviral Epigenetic Library: In Vivo Screening. The in vitro mutagenesis and in vivo screening approaches were modified based known methods (Huang et al. (2021) Cell 184:1245-1261 e1221). Briefly, retrovirus was produced by co-transfecting the retroviral epigenetic library plasmid with packaging vector (pCL-Eco) in Plat-E cells. At 48 hours after transfection, the supernatant was harvested and frozen at −80° C. Naïve Cas9-expressing OT-I cells were isolated from two Cas9-OT-I mice and activated overnight with plate-bound anti-CD3ε (10 μg/ml; Bio-X-Cell) and anti-CD28 (5 μg/ml; Bio-X-Cell) antibodies. After activation, T cells were transduced with the retrovirus library at low multiplicity of infection to achieve ˜20% transduction efficiency. Cells were washed and cultured in media supplemented with murine IL-7 (12.5 ng/ml; Peprotech) and IL-15 (25 ng/ml; PeproTech) for 4 days to expand and allow gene editing to occur. Transduced cells were sorted based on the expression of Ametrine and an aliquot of 0.5×10⁶ transduced OT-I cells was saved as “day 0 input” (>200× cell coverage per sgRNA). Transduced OT-I cells (0.2×10⁶) were then transferred i.v. to twenty-one Cas9 expressing hosts followed by Lm-Ova infection (3×10⁴ CFU) 2 hours later. At day 7.5 or 36 after infection, total donor-derived cells, terminal effector (TE, KLRG1^{hi}CD127^{lo}) and memory precursor (MP, KLRG1^{hi}CD127^{lo}) cells were sorted and frozen at −80° C. until genomic DNA extraction. A minimum of 0.5×10⁶ OT-I cells per sample (>200× cell coverage per sgRNA) was recovered for further analysis.

Library Preparation. Genomic DNA was extracted by using the DNeasy Blood & Tissue Kits (Qiagen) according to the manufacturer's instruction. Two rounds of PCR were performed by using the KOD Hot Start DNA Polymerase (Sigma-Aldrich) with primary PCR to amplify the sgRNAs and second PCR to attach Illumina NEXTERA® adapters to each sample. PCR products were purified using AMPURE® XP beads (Beckman Coulter) after each PCR reaction. Primers sequences to amplify sgRNAs for the first PCR were: NEXTERA® NGS-F: TCG GCA GCG TCA GAT GTG TAT AAG AGA CAG TTG TGG AAA GGA CGA AAC ACC G (SEQ ID NO:2); NEXTERA® NGS-R: GTC TCG TGG GCT CGG AGA TGT GTA TAA GAG ACA GCC ACT TTT TCA AGT TGA TAA CGG (SEQ ID NO: 3). The library sequencing was performed in HISEQ® (Illumina) platform.

Data Processing of In Vivo Pooled CRISPR Screening. For data analysis, raw FASTQ files obtained after sequencing were demultiplexed using the HISEQ® Analysis software (Illumina). Single-end reads were trimmed and quality-filtered using the CLC Genomics Workbench v.11 (Qiagen) and matched against sgRNA sequences from the sgRNA epigenetic library. Read counts for sgRNAs were normalized against total read counts across all samples. For each sgRNA, the fold change (log 2-transformed ratio) for enrichment was calculated between each of the biological replicates and the input experiment. The gene-level false-discovery-rate (FDR)-adjusted p value was calculated among multiple sgRNAs (n=6) of each gene, using a two-tailed paired Student's t-test between log 2-transformed average normalized read counts of MP samples and those of TE samples, between counts of total day 7.5 samples and those of total day 36 samples.

Tissue Dissociation of Non-Lymphoid Organs. Lung and liver were collected and minced into small pieces using razor blades. The organs were digested in dissociation buffer containing 1 mg/ml of collagenase IV (Worthington Biochemicals) and 0.5 mg/ml of DNase I (Sigma-Aldrich) at 37° C. for 30 minutes on a 3D orbital mixer. The cell suspensions were then passed through 70-μm filters to remove undigested tissues and followed by density-gradient centrifugation over colloidal silica particles sold under the tradename PERCOLL® (GE Healthcare).

Flow Cytometry. Antibodies from Biolegend included APC/Cy7 anti-human CD8α, BV785 anti-human CD45RA, PE anti-human CD62L, APC anti-human CD27, PE/Cy7 anti-human CCR7, BV421 anti-human CD95, BV785 anti-mouse/human CD44, BV510 anti-mouse KLRG1, KIRAVIA Blue 520 anti-mouse CD62L, BV605 anti-mouse CD127, BV650 anti-mouse CX3CR1, APC anti-mouse CD8α, ALEXA FLUOR® 700 fluorescent dye anti-mouse CD45.2, APC anti-mouse CD98; PE anti-mouse CXCR3 (eBioscience); and BUV395 anti-human CD3, BUV805 anti-human CD45RO; BUV496 anti-mouse CD45.1, BUV805 anti-mouse CD8α were acquired from BD Biosciences. For surface markers, cells were stained for 30 minutes at 4° C. in PBS+2% (w/v) bovine serum albumin (BSA). To examine intracellular cytokine production, splenocytes were stimulated with 1 μg/ml ovalbumin peptide (SIINFEKL; SEQ ID NO: 1) in the presence of monensin (BD Biosciences) for 5 hours and stained with anti-IFN-γ (BioLegend), anti-TNF-α (Thermo Fisher Scientific) and anti-Granzyme B (BioLegend) using a fixation/permeabilization kit (BD Biosciences). Fixable viability dye (Thermo Fisher Scientific) was used for dead-cell exclusion. Samples were acquired on CYTEK® Aurora or BD LSRII flow cytometer and analyzed with FlowJo software.

In Vivo Tumor Transplant Experiment. For B16-Ova and MC38-Ova tumors, 0.5×10⁶ tumor cells were injected subcutaneously into naïve mice (7-10 weeks of age). OT-I cells (2×10⁶) were transferred i.v. 7 days after tumor injection. Tumors were measured every two days.

For tumor re-challenge assays, sgNTC or indicated sgRNA-transduced OT-I cells (1×10⁴) were transferred into Cas9-expressing mice (7-10 weeks of age) followed by 3×10⁴ CFU of Lm-Ova infection. At >30 days post-infection, B16-Ova cells (1×10⁷) were injected subcutaneously. Tumor size was measured every three days.

For CAR-T tumor models, the murine osteosarcoma cell line F420 was derived from singly floxed p53+/F-Col2.3 transgenic mice (Zhao et al. (2015) Oncogene 34:5069-5079); 0.5×10⁶ F420 cells were injected subcutaneously into naïve mice. Seven days after tumor transplant, 5×10⁶ B7-H3 CAR-T cells were transferred i.v. without lymphodepleting chemotherapy and tumor size was measured every two days.

Generation of Murine CAR-T Cells. Generation of murine (m) B7-H3-CAR constructs have been previously described (Haydar et al. (2021) Neuro-Oncol. 23:999-1011). Briefly, a codon-optimized DNA was generated encoding mB7-H3-specific scFv derived from the m276 monoclonal antibody (Seaman et al. (2017) Cancer Cell 31:501-515 e508) followed by a portion of the murine CD28 molecule (including CD28 extracellular, transmembrane, and cytoplasmic domains) and a CD3ζ domain (with mutated and 1^{st} and 3^{rd} immunoreceptor tyrosine-based activation motifs (ITAMs)) as described before (Kochenderfer et al. (2010) Blood 116:3875-3886). The sequence was synthesized by GENEART® (Thermo Fisher) and ligated using cut-and-paste cloning into the mouse stem cell virus-based splice-gag vector (MSGV) retroviral backbone between HindIII and SacII cutting sites replacing the ID3-28Z CAR sequence (Kochenderfer et al. (2010) Blood 116:3875-3886) with mB7-H3.CD28.CD3ζ sequence. The sequence of final mB7-H3. CD28.ζ CAR construct was verified by sequencing. As a control, a mB7-H3 STOP-CAR was generated where CD28.CD3ζ endodomain has been deleted from mB7-H3-CAR using In-Fusion cloning. The sequence of the STOP-CAR construct was verified by sequencing.

The retrovirus for murine CAR-T cell generation was produced in accordance with known methods (Haydar et al. (2021) Neuro-Oncol. 23:999-1011). Briefly, retroviral particles were generated by transient transfection of 293T cells with the CAR-encoding plasmid, Peg-Pam plasmid encoding MoMLV gag-pol, and plasmid encoding the VSVG envelope. Virus was harvested at 48 hours and filtered with 0.45 mm filter. The VSVG-pseudo typed virus was then used to transduce the GPE86 producer cell line. B7-H3-CAR-expressing GPE86 cells were stained with fluorescent dye sold under the tradename ALEXA FLUOR®-647 anti-human IgG, F(ab′)₂ fragment antibody (Jackson ImmunoResearch) and sorted by using BD FACSAria III.

Murine CAR-T cells were generated as described before (Haydar et al. (2021) Neuro-Oncol. 23:999-1011) with modification. Naïve CD8⁺ T cells from 6-8 weeks old CD45.1⁺ mice were activated with plate bound anti-CD3ε (1 μg/ml, Bio-X-Cell), anti-CD28 (2 μg/ml, Bio-X-Cell) and 50 U/ml rhIL-2 (Peprotech). On day 2 post-activation, CD8⁺ T cells were transduced with retrovirus expressing B7-H3-CAR or control CAR on recombinant human fibronectin sold under the tradename RETRONECTIN® (Takara)-coated non-tissue culture treated plate in complete RPMI medium supplemented with 50 U/ml rhIL-2. At 48 hours after transduction, CAR-T cells were harvested and expanded in the presence of 10 ng/ml rhIL-15 (Peprotech) for another 3 days and then used d for in vivo and in vitro experiments.

Repeat Stimulation Assay. For repeat stimulation assays, 3×10⁵ tumor cells (GL261, F420) were plated in a 24-well plate and allowed to adhere for 4-6 hours at 37° C., CAR-T cells (1×10⁶) were then added to the wells containing tumor cells without any exogenous cytokines (tumor: CAR-T cell ratio=1:3). After 3 days of co-culture, CAR-T cells were lifted by gentle pipetting to avoid disrupting adherent tumor cells, and the number of T cells was determined using a hemocytometer. Stimulations and cell counts were repeated every 3 days with the same tumor cells.

Co-Immunoprecipitation. For endogenous CO-immunoprecipitation, naïve CD8⁺ T cells were stimulated on plate-bound anti-CD3ε (2 μg/ml, Bio-X-Cell), anti-CD28 (1 μg/ml, Bio-X-Cell) and ICAM1 (0.5 μg/ml, Peprotech) for 36 hours. Nuclear extracts were prepared as described (Valencia et al. (2019) Cell 179:1342-1356 e1323) with some modifications. Briefly, cells were harvested and resuspended in hypotonic buffer (50 mM Tris, pH7.5, 0.1% NP-40, 1 mM MgCl₂ supplemented with complete, EDTA-free Protease Inhibitor Cocktail (Roche). Nuclei were pelleted at 5000 rpm for 10 minutes at 4° C. and resuspended in CelLytic M buffer (Sigma). Lysates were incubated at 4° C. for 30 minutes and pelleted at 20,000 g for 10 minutes at 4° C. Supernatants were collected for co-immunoprecipitation. Nuclear extracts were incubated at 4° C. overnight with 2 mg of the following antibodies: anti-Arid1a (Cell Signaling Technology), anti-c-Myc (Cell Signaling Technology) and rabbit mAb IgG XP Isotype Control (Cell Signaling Technology). Samples were then incubated with Dynabeads protein A (10001D, Thermo Fisher Scientific) for 1 hour at 4° C. Beads were washed three times in RIPA buffer and eluted with 1× sample buffer (Bio-Rad).

Immunoblot. Cells were lysed in CelLytic M buffer (Sigma). Cell lysates and immunoprecipitated complexes were separated by and blotted SDS-PAGE with the following antibodies: anti-Arid1a (1:1000; Cell Signaling Technology), anti-Brg1 (1:1000; Abcam), anti-Brg1 (1:1000; Santa Cruz), anti-c-Myc, anti-Smarcb1 (1:1000; Abcam), anti-Pbrm1 (1:1000; Millipore), anti-Brm (1:1000; Abcam), anti-Smarcd1 (1:1000; Bethyl), anti-Smarcd2 (1:1000; Bethyl), anti-Baf57 (1:1000; Abcam), anti-Baf155 (1:1000; Cell Signaling Technology), anti-Baf170 (1:1000; Cell Signaling Technology), anti-Baf45c (1:1000; ThermoFisher Scientific), anti-Baf45d (1:1000; Bethyl), anti-Brd7 (1000; Santa Cruz), anti-Brd9 (1:1000; Active Motif), anti-Actin (1:1000; Santa Cruz), anti-Gapdh (1:1000; MAB374, Millipore).

Immunofluorescence Staining and Imaging. Naïve CD8⁺ T cells were stimulated on plate-bound anti-CD3ε (2 μg/ml, Bio-X-Cell), anti-CD28 (1 μg/ml, Bio-X-Cell) and ICAM1 (0.5 μg/ml, Peprotech) for 28 hours followed by fixation for 10 minutes with 4% paraformaldehyde (PFA) (Electron Microscopy Science) at room temperature (RT). Cells were rinsed with TBS (50 mM Tris pH 8.0, 100 mM NaCl) and permeabilized with permeabilization buffer (Tris pH 8.0, 100 mM NaCl, 0.3% (v/v) TRITON X-100) for 5 minutes at RT, and then blocked with TBS+2% BSA (Sigma) for 30 minutes at RT. Cells were stained at 4° C. overnight with the following primary antibodies: anti-c-Myc (1:500; Cell Signaling Technology), anti-Arid1a (1:500; Abcam), anti-Brg1 (1:500; Abcam), anti-Smarcb1 (1:250; Abcam), anti-tubulin (1:1000; Sigma), anti-tubulin (1:1000; Invitrogen), anti-Lamin B1 (1:500; Abcam). Samples were washed twice with TBS and incubated with for 1 hour at RT with the following secondary anti-bodies: anti-rabbit ALEXA FLUOR® fluorescent dye plus 555, anti-mouse Alexa Fluor plus 647 (1:2000; Thermo Fisher Scientific), anti-rat ALEXA FLUOR® 647 fluorescent dye (1:1000; Jackson ImmunoResearch), anti-rabbit ALEXA FLUOR® fluorescent dye plus 555 (1:2000; Thermo Fisher Scientific). Samples were imaged by Marianas spin disk confocal (Intelligent Imaging Innovations), Prime95B sCMOS camera, and 405, 488, 561 and 640 nm laser lines were used. Dividing sister cells were identified by the presence of an intercellular cytokinetic bridge based on tubulin staining. Sum fluorescent intensities were analyzed by Slide book 6 (Intelligent Imaging Innovations) and Imaris (Oxford Instruments).

ATAC-see. ATCC-see was performed as described (Chen et al. (2016) Nat. Methods 13:1013-1020). Briefly, naïve CD8⁺ T cells were stimulated with anti-CD3/28 and ICAM1 for 28 hours followed by fixation for 10 minutes at RT with 4% PFA. Cells were permeabilized with lysis buffer (10 mM Tris, pH 7.4, 10 mM NaCl, 3 mM MgCl₂, 0.01% NP-40) for 10 minutes at RT. Samples were washed twice with PBS in a humid chamber box at 37° C. The transposase solution (150 ml 2×TD buffer, 100 nM Tn5-ATTO550N, add H₂O to 300 ml) was added to the chambered cover glass and incubated for 30 minutes at 37° C. Then, samples were washed three times with stop buffer (0.01% SDS, 50 mM EDTA in PBS) for 15 minutes at 55° C. Immunofluorescence staining was performed after ATAC-see staining.

Microarray Analysis. A total of 1×10⁵ donor-derived OT-I cells were sorted, and RNA was isolated using the RNeasy Micro Kit (Qiagen) following the manufacturer's instructions. The RNA Integrity Number (RIN) and concentration of RNA were measured by Agilent 2100 bioanalyzer. RNA (1 ng) was used for subsequent microarray analysis with Clariom S mouse array platform (Thermo Fisher Scientific). To perform microarray analyses, the gene expression signals were summarized with the robust multi-array average algorithm (Affymetrix Expression Console v1.1). The differentially expressed gene analysis was performed using lmFit method implemented in R package limma v.3.34.9 (Ritchie et al. (2015) Nucl. Acids Res. 43: e47). FDR was calculated by Benjamini-Hochberg method. Plots were generated using the R package ggplot2 v.2.2.1. Gene set enrichment analysis (GSEA) and functional gene set enrichment using C7 immunological collections from the Molecular Signatures Database was performed as previously described (Subramanian et al. (2005) Proc. Natl. Acad. Sci. USA 102:15545-15550). Several gene sets that were frequently used in this study were renamed as follows: MP and T signatures, which are respectively “IL-7R low vs high eff CD8 T cell DN” and “IL-7R low vs high eff CD8 T cell UP” signatures (Joshi et al. (2007) Immunity 27:281-295).

ATAC-seq. Naïve CD8⁺ T cells were activated with anti-CD3/CD28 plus ICAM1 for 36 hours and first division wild-type (WT) CD8⁺ T cells or CD8⁺ T cells with acute c-Myc deletion (for c-Myc acute knockout, naïve Myc^{fl/fl} Rosa26^{Cre-ERT2} CD8⁺ T cells were treated with 4OHT (4-Hydroxytamoxifen) (Sigma-Aldrich) overnight with 50 U/ml rhIL-2 before activation) were sorted into medium and processed to generate ATAC-seq libraries immediately as previously described (Buenrostro et al. (2015) Curr. Protoc. Mol. Biol. 109 (1): 21.29.1-21.29.9) with slight modifications. Briefly, 50,000 cells were pelleted at 500 g for 5 minutes at 4° C. Samples were washed in 1 ml cold PBS and resuspended in 50 ml cold cell lysis buffer (10 mM Tris pH 7.4, 10 mM NaCl, 3 mM MgCl₂, 0.1% NP-40). Nuclei were pelleted at 1000 g for 10 minutes and resuspended in 50 ml transposition reaction mixture (25 ml TD buffer, 2.5 ml TDE1, 22.5 ml ddH₂O) (Illumina). Samples were incubated at 42° C. for 45 minutes. DNA was then purified using MiniElute PCR Purification Kit (Qiagen). Libraries were barcoded and amplified with 05 High-Fidelity 2×Master Mix (NEB). Libraries were quantified using Qubit and size distribution determined by Agilent 4200 TapeStation before sequencing.

Analysis for ATAC-seq Data. Paired-end sequencing reads were trimmed with Trim Galore (version 0.5.0) with default parameters. The reads were then aligned to the reference mouse mm10 assembly using Bowtie 2 (version 2.3.5.1) with settings—end-to-end—very-sensitive −X 2000. The resulting alignments, recorded in BAM file, were sorted, indexed, and marked for duplicates with Picard MarkDuplicates function (version 2.19.0). Afterward, the BAM file was filtered with SAMtools (version 1.9), BamTools (version 2.5.1), and scripts of nf-core/chipseq (Ewels et al. (2020) Nat. Biotechnol. 38:276-278) to discard reads, mates that were unmapped, duplicates, PCR/optical not primary alignments, mapped to multiple locations, or mapped to ENCODE blacklisted regions (Amemiya et al. (2019) Sci. Rep. 9:9354); only reads mapped in proper pair were kept (−F 1804 −f 2 −q 30). MACS (version 2.1.2) was used to call peaks from the BAM file with narrowPeak setting, —extsize 200, and recommended mappable genome size (default value for other parameters). Chromatin accessibility signal was normalized by scaling to 1 million mapped reads using BEDTools (version 2.27.1) and bedGraphToBigWig (version 377), and visualized as heatmaps using deepTools plotHeatmap (version 3.2.1).

In ATAC-seq experiments with spike-in Drosophila S2 cells, two modifications were made from above analysis: i) a hybrid reference of mouse mm10 and Drosophila melanogaster Ensembl r97 was used, ii) signals were normalized by scaling to per million reads mapped to Drosophila. Differentially accessibility analysis was performed using DiffBind (version 2.16.0) (summit=250, DESeq2 and other default parameters). Peaks were annotated to the nearest genes with annotatePeak function in R package ChIPseeker (version 1.26.2) at gene level with default parameters. Genes were ranked by Wald statistic from DESeq2 (if a gene was associated with multiple peaks, the mean was used). Gene set enrichment analysis (GSEA) was then performed against MSigDB, GO, Reactome, KEGG with R package clusterProfiler (version 3.18.1).

CUT&RUN ChIP-seq. CUT&RUN ChIP-seq experiments were performed as previously described (Meers et al. (2019) Elife 8: e46314) with slight modifications. For first division CD8⁺ T cell samples, naïve CD8⁺ T cells were stimulated on plate-bound anti-CD3/CD28 plus ICAM1 for 36 hours and first-division CD8⁺ T cells were sorted. For c-Myc knockout samples, naïve CD8⁺ T cells from Myc^{fl/fl}Rosa26^{Cre-ERT2} mice were treated with 4OHT overnight before stimulation; dead cells were removed by using Dead Cell Removal Kit (Miltenyi Biotec). Cells (3×10⁵) were washed with wash (20 mM HEPES (Sigma-Aldrich), 150 mM NaCl (Invitrogen), 0.5 mM spermidine (Sigma-Aldrich), and protease inhibitor cocktail (Sigma-Aldrich)) twice. Cells were then resuspended and bound to concanavalin A-coated magnetic beads (Bang Laboratories). Samples were placed on the magnetic stand, residual liquid was removed from beads and beads were resuspended in 100 μl antibody buffer (20 mM HEPES, 150 mM NaCl, 0.5 mM spermidine, 0.01% digitonin (Millipore), 2 mM EDTA (Invitrogen), and Protease inhibitor cocktail). Primary antibodies were added to the samples at a final concentration of 1:100 and incubated overnight at 4° C. The next day, samples were washed with cold Dig-wash buffer (20 mM HEPES, 150 mM NaCl, 0.5 mM spermidine, 0.01% digitonin and protease inhibitor) twice. After washing, pAG-MNase (Addgene) was added to tubes and rotated at 4° C. for 1 hour. After two washes with Dig-wash buffer, samples were resuspended in 50 μl Dig-wash buffer. Two μl of 100 mM CaCl₂) (Sigma-Aldrich) was added per sample, briefly vortexed, and immediately placed on ice for 30 minutes. Fifty μl 2×STOP buffer [340 mM NaCl, 20 mM EDTA, 4 mM EGTA (Sigma-Aldrich), 100 μg/mL RNase A (Thermo-Fisher), and 50 μg/mL GlycoBlue (Invitrogen)] were added and mixed by gentle vertexing. Then, samples were incubated for 30 minutes at 37° C. to release CUT&RUN fragments. Fragmented DNA was purified with the NEB Monarch PCR&DNA Cleanup Kit (NEB). DNA libraries were prepared using the NEBNext Ultra II DNA Library Prep Kit (NEB) and purified with AMPure SPRI beads (Beckman-Coulter). Libraries were quantified using Qubit and size distribution was determined by Agilent 4200 TapeStation analysis before 15 million paired-end sequencing was performed. Peaks were annotated to the nearest genes with annotatePeak function in R package ChIPseeker (version 1.26.2) at gene level with default parameters. Enrichment analysis was performed against MSigDB, GO, Reactome, KEGG with the over-representation approach implemented in R package clusterProfiler (version 3.18.1).

CUT&RUN Data Processing. Paired-end sequencing reads were trimmed with Trim Galore (version 0.5.0) with default parameters. Then, reads were aligned to the reference mouse mm10 assembly using Bowtie 2 (version 2.3.5.1) with settings—end-to-end—very-sensitive—no-mixed—no-discordant −q —phred33 −I 10 −X 700. The resulting alignments, recorded in BAM file, were sorted, indexed, and marked for duplicates with Picard MarkDuplicates function (version 2.19.0). Afterward, the BAM file was filtered with SAMtools (version 1.9), BamTools (version 2.5.1), and scripts of nf948 core/chipseq (Ewels et al. (2020) Nat. Biotechnol. 38:276-278) to discard reads, mates that were unmapped, PCR/optical duplicates, not primary alignments, mapped to multiple locations, mapped to ENCODE blacklisted regions (Amemiya et al. (2019) Sci. Rep. 9:9354), or have more than four mismatches (−F 0x004 −F 0x008 −F 0x0100 −F 0x0400 −f 0x001 −q 1). MACS (version 2.1.2) was used to call peaks from the BAM file with IgG control and recommended mappable genome size (default value for other parameters). NarrowPeak mode was used for c-Myc, while broadPeak mode was used for Aridla and Brg1. Binding signal was normalized by scaling to one million mapped reads using BEDTools (version 2.27.1) and bedGraphToBigWig (version 377) and visualized as heatmaps using deepTools plotHeatmap (version 3.2.1). In CUT&RUN experiments with spike-in E. coli, two modifications were made: i) a hybrid reference of mouse mm10 and E. coli ASM584v2 was used, ii) signals were normalized by scaling to per million reads mapped to E. coli.

RNA-seq. For first division CD8⁺ T cell samples, naive CD8⁺ T cells were stimulated on plate-bound anti-CD3/CD28 plus ICAM1 for 36 hours and first-division CD8⁺ T cells were sorted. For c-Myc acute knockout samples, naïve CD8⁺ T cells from Myc^{fl/fl}Rosa26^{Cre-ERT2} mice were treated with 4OHT overnight before stimulation; dead cells were removed by using Dead Cell Removal Kit (Miltenyi Biotec). Total RNA was isolated using Direct-zol RNA Microprep Kits (Zymo Research) following the manufacturer's instruction and quantified using Agilent 4200 TapeStation. Libraries were prepared using KAPA RNA HyperPrep Kit with RiboErase (HMR) (08098131702, Roche) and purified by AMPure SPRI beads (Beckman-Coulter). Libraries were quantified and size distribution was determined by Agilent 4200 Tapestation before paired-end sequencing was performed.

RNA-seq Data Processing. Paired-end sequencing reads were mapped by the pipeline of the St Jude Center for Applied Bioinformatics. Briefly, the reads were trimmed with Trim Galore (version 0.5.0) with default parameters. Then, reads were aligned to the reference mouse mm10 assembly plus ERCC spike in sequences using STAR (version 2.7.5a). The resulting alignments, recorded in BAM file, were sorted, indexed, and marked for duplicates with Picard MarkDuplicates function (version 2.19.0). Transcript quantification was calculated using RSEM (Li & Dewey (2011) BMC Bioinformatics 12:323). Differential gene expression analysis was carried out with DESeq2 with Wald test (default parameters) (Love et al. (2014) Genome Biol. 15:550). Genes were pre-filtered to have at least five reads in at least two samples. Gene set enrichment analysis (GSEA) was performed against MSigDB, GO, Reactome, KEGG with R package clusterProfiler (version 3.18.1). Wald statistic calculated from DESeq2 was used for gene ranking.

For the RNA-seq experiment with ERCC spike-in. RUVg function in RUVSeq package was used for normalization followed by DESeq2 according to RUVSeq manual (Risso et al. (2014) Nat. Biotechnol. 32:896-902).

Quantification and Statistical Analysis. Data are plotted and analyzed by GraphPad Prism (GraphPad Software, version 9.2.0). Statistical significance was calculated with unpaired or paired two-tailed Student's t-test. Two-way ANOVA was performed to compare tumor growth curves. The log-rank (Mantel-Cox) test was performed for comparing mouse survival curves. P value less than 0.05 was considered significant. Data are presented as mean±s.e.m.

Example 2: The SWI/SNF Canonical BAF Complex and c-Myc Cooperate to Promote Early Fate Decisions in CD8⁺ T Cells

A pooled CRISPR screen was employed for identifying potential inhibitors of antigen-specific T_{MEM} cell generation in vivo (Huang et al. (2021) Cell 184:1245-1261). In this approach, a guide RNA (sgRNA) library was designed to target proteins involved in epigenetic modification. After adoptive transfer of sgRNA-expressing ovalbumin (Ova)-specific CD8⁺ T (OT-I) cells and challenge with Listeria monocytogenes expressing Ova (Lm-Ova), comparative analysis of sgRNAs was performed in memory precursor (MP; KLRG1^{lo}CD127^{hi}) versus terminal effector (TE; KLRG1^{hi}CD127^{lo}) (Joshi et al. (2007) Immunity 27:281-343; Omilusik & Goldrath (2019) Curr. Opin. Immunol. 58:89-97) CD8⁺ T cells at day 7.5, and CD8⁺ T cell accumulation at 36 days (memory phase) versus 7.5 days (effector phase). These analyses revealed a striking enrichment for sgRNAs targeting several components of the cBAF complex including Aridla, Smarcc1, Smarcc2, Smarcd2, and Smarca4. To validate these findings, the cBAF components Smarcd2 or Arid1a were ablated via CRISPR editing in OT-I cells. After adoptive transfer, the recipient mice were challenged with Lm-Ova in vivo. Deletion of either component resulted in decreased TE and increased MP cells when assessed on day 7.5, as reflected by reduced proportion of MP versus TE cells (MP/TE ratio), reduced expression of TE-associated molecules (e.g., CX3CR1, KLRG1) (Omilusik & Goldrath (2019) Curr. Opin. Immunol. 58:89-97; Kaech & Cui (2012) Nat. Rev. Immunol. 12:749-761) and increased memory-associated molecules (e.g., CXCR3, CD27, and CD62L) (Omilusik & Goldrath (2019) Curr. Opin. Immunol. 58:89-97; Kaech & Cui (2012) Nat. Rev. Immunol. 12:749-761), indicating a pro-memory cell fate.

Consistent with these findings, transcriptional profiling of Smarcd2 or Arid1a-deficient T cells at day 7.5 post-infection revealed enrichment of a gene expression signature of MP cells and a reduction in expression of a TE cell signature (Joshi et al. (2007) Immunity 27:281-343) in Smarcd2- or Arid1a-deficient cells. Further, Arid1a^{fl/fl} mice expressing Rosa26^{Cre-ERT2} and deleted Arid1a were generated by oral treatment with tamoxifen, followed by activation of CD8⁺ T cells with anti-CD38 and anti-CD28 (anti-CD3/CD28) plus ICAM1 in vitro. Acute deletion of Arid1a enhanced a T_{MEM}-like gene program and suppressed a T_{EFF}-like gene signature following the first cell division in vitro, as revealed by transcriptional profiling. Upon adoptive transfer of Arid1a-deficient OT-I cells into recipient mice and IAV-Ova infection, increased numbers and percentages of MP cells were observed in the absence of Arid1a, further supporting the pro-memory fate decision upon deletion of cBAF components.

Memory responses at day >30 post-infection were subsequently analyzed. Specifically, cells were transduced with sgRNA targeting Smarcd2 or Arid1a. Following adoptive transfer and Lm-Ova infection, increased enrichment of T_{MEM} cells were observed in diverse tissues during the memory phase (day >30 post-infection). These results indicate an enhanced T_{MEM} generation upon deletion of Smarcd2 or Arid1a. To determine if cBAF-deficient T cells displayed bona fide memory features, T_{MEM} cells were sorted from the donor mice that had been infected with Lm-Ova and the T_{MEM} cells were transferred into (i) lymphocyte-deficient Rag1^{-/-} animals to assess homeostatic proliferation (Wherry et al. (2003) Nat. Immunol. 4:225-234), or (ii) naïve hosts for secondary rechallenge with Lm-Ova in vivo. Compared to wild-type (WT) T cells transduced with non-targeting control (NTC), Smarcd2 or Arid1a-deficient T cells displayed increased homeostatic proliferation in Rag1^{-/-} hosts. Upon transfer into naive recipients and rechallenge, loss of CBAF resulted in increased numbers of cells expressing interferon-γ (IFN-γ), TNF-α, and Granzyme B (GzmB). Therefore, deletion of cBAF components promotes T_{MEM} generation and function.

cBAF and polybromo-associated BAF (PBAF) constitute two major mammalian SWI/SNF complexes, which are composed of common and unique components (Centore et al. (2020) Trends Genet. 36:936-950; Mittal & Roberts (2020) Nat. Rev. Clin. Oncol. 17:435-448). As PBAF shares many common subunits with CBAF, the contribution of PBAF and cBAF complexes to T_{MEM} formation was analyzed. Pbrm1 (also called BAF180) is present in PBAF but not cBAF (Centore et al. (2020) Trends Genet. 36:936-950; Mittal & Roberts (2020) Nat. Rev. Clin. Oncol. 17:435-448; Mashtalir et al. (2018) Cell 175:1272-1288). Ablation of Pbrm1 in Pbrm1^{fl/fl}Cd4-Cre CD8⁺ T cells had no effect on total CD8⁺ T cells, or TE or MP generation at 7.5 or 32 days following Lm-Ova infection. Pbrm1 deficiency also did not affect Ova-specific CD8⁺ T cell numbers in different tissues or expression of CD62L, the canonical marker of central memory T (TCM) cells (Omilusik & Goldrath (2019) Curr. Opin. Immunol. 58:89-97; Kaech & Cui (2012) Nat. Rev. Immunol. 12:749-761). In contrast, deletion of the components Brg1 or Smarcc1 (present in both cBAF and PBAF complexes) promoted MP generation and suppressed TE differentiation, as reflected by increased proportions of CD62L⁺ and KLRG1^{lo}CD127^{hi} cells and decreased proportions of KLRG1^{hi}CD127^{lo} cells. cBAF contains one of two mutually exclusive catalytic subunits, Brg1 or Brm (also known as Smarca2; Centore et al. (2020) Trends Genet. 36:936-950; Mittal & Roberts (2020) Nat. Rev. Clin. Oncol. 17:435-448). In contrast to Brg1, sgRNA targeting of Brm had no effect on TE or MP differentiation. Altogether, it was concluded that the Brg1-containing CBAF complex is functionally important to promote TE and suppress T_{MEM} generation in activated CD8⁺ T cells.

Similar to the enrichment of sgRNAs targeting the cBAF complex, the screen revealed an enrichment for sgRNAs targeting the Myc protooncogene in MP versus TE and day 36 T_{MEM} Versus day 7.5 T_{EFF} cells. It was previously found that ablation of one allele of c-Myc can promote the differentiation of activated CD8⁺ T cells to a memory-like phenotype, and that c-Myc protein is sorted asymmetrically into the two daughter cells during the first cell division (Verbist et al. (2016) Nature 532:389-393). Therefore, the relationships between c-Myc protein expression and expression of components of the cBAF complex were examined at early time points following TCR stimulation. OT-I T cells that expressed GFP-tagged c-Myc (GFP inserted onto the C-terminus of c-Myc; Verbist et al. (2016) Nature 532:389-393; Huang et al. (2008) Eur. J. Immunol. 38:342-349) were activated with anti-CD3/CD28 plus ICAM1, and it was observed, via confocal microscopy, that telophase cells were completing the first cell division. In dividing T cells with asymmetric distribution of c-Myc in the telophase nuclei, a corresponding symmetric distribution of the cBAF components, Smarcb1 (R²=0.3814, p=0.0013), Arid1a (R²=0.5199, p=0.0081), and Brg1 (R²=0.6007, p=0.0142) was found. Sorting of first-division CD8⁺ T cells based on expression of c-Myc-GFP for immunoblot analysis similarly revealed increased components of the cBAF complex in c-Myc^{hi} versus c-Myc^{lo} cells, including Smarcd2 and Arid1a. Asymmetric expression of CD98 correlates with c-Myc expression in first-division CD8⁺ T cells (Verbist et al. (2016) Nature 532:389-393), and increased cBAF components was similarly observed in CD98^{hi} versus CD98^{lo} first-division CD8⁺ T cells. Therefore, like c-Myc, cBAF shows asymmetric distribution at the first division of CD8⁺ T cells.

The BAF complexes are important for controlling chromatin architecture through nucleosome mobilization, ejection, and histone dimer exchange (Mittal & Roberts (2020) Nat. Rev. Clin. Oncol. 17:435-448; Clapier et al. (2017) Nat. Rev. Mol. Cell. Biol. 18:407-422; Kassabov et al. (2003) Mol. Cell 11:391-403; Kwon et a al. (1994) Nature 370:477-481; Hargreaves et al. (2011) Cell Res. 21:396-420). Therefore, the accessible genome during the first division of CD8+ T cells was visualized using transposase-accessible chromatin with visualization (ATAC-see), a transposase-mediated technology that directly images chromatin accessibility in situ (Chen et al. (2016) Nat. Methods 13:1013-1020). In line with the observations of asymmetric assortment of cBAF components, many first division sister cells were observed that differed in the intensity of ATAC-see, indicating various degrees of chromatin openness. An assay for transposase-accessible chromatin was subsequently performed with high-throughput sequencing (ATAC-seq) using sorted, first-division c-Myc^{hi} and c-Myc^{lo} CD8⁺ T cells. Extensive differences in chromatin accessibility were observed between these populations, with more open accessibility detected in the c-Myc^{hi} cells in the promoter, intronic, and intergenic regions. Also, gene set enrichment analysis of the genes closest to differentially accessible regions revealed gene signatures consistent with effector versus memory T cells, with c-Myc^{lo} cells and c-Myc^{hi} cells exhibiting memory-like and effector-like signatures, respectively. These results are consistent with the higher assortment of cBAF in c-Myc^{hi} cells described above. Thus, chromatin accessibility and cBAF expression are coordinately regulated, with higher activities observed in c-Myc^{hi} cells.

First-division c-Myc^{hi} CD8⁺ T cells display effector-like function in vivo, while c-Myc^{lo} cells display a memory-like function (Verbist et al. (2016) Nature 532:389-393). To examine whether this process is dependent upon cBAF function, OT-I cells from Arid1a^{fl/fl}; Rosa26^{Cre-ERT2} mice were activated and first-division c-Myc^{hi} and c-Myc^{lo} cells were sorted. Arid1a expression was ablated in the sorted cells by treatment with 4-Hydroxytamoxifen (4OHT) before adoptive transfer into recipient mice and infection with IAV-Ova. At 9 days post-infection, OT-I cells were examined for markers of TE and MP. Cells that had been c-Myc^{hi} at sorting generated more cells with markers of TE cells than those that had been c-Myc^{lo} at sorting, and conversely, the latter generated more cells with MP markers. Ablation of Arid1a following the sort eliminated this effect; all cells (originally c-Myc^{hi or lo}) showed a tendency to differentiate into MP, indicating the functional requirement of cBAF for T cell fate decisions associated with c-Myc expression levels.

These observations prompted an examination of the functional interaction between cBAF and c-Myc. It was found that Arid1a and Smarcb1 co-precipitated with c-Myc, and reciprocally, c-Myc and Smarcb1 co-precipitated with Arid1a in activated CD8⁺ T cells, as described for other cell types (Sammak et al. (2018) FEBS J. 285:4165-4180; Cheng et al. (1999) Nat. Genet. 22:102-105; Stojanova et al. (2016) Cell Cycle 15:1693-1705). Chromatin binding of c-Myc, Arid1a, and Brg1 in first division CD8⁺ T cells was subsequently examined after anti-CD3/CD28 plus ICAM1 stimulation for 36 hours using the CUT & RUN (an alternative to chromatin assay immunoprecipitation sequencing (ChIP-seq) for low-input materials; Meers et al. (2019) Elife 8: e46314). This analysis indicated that more than 80% of the peaks overlapped between Arid1a and Brg1. Importantly, a substantial proportion of Arid1a (45%) and Brg1 (42%) binding sites were occupied by c-Myc. Gene set enrichment analysis of binding sites occupied by both c-Myc and Arid1a, or c-Myc and Brg1 revealed the expected Myc target gene sets and also gene sets involved in T cell activation and differentiation and many effector-associated molecules. Moreover, the binding of Arid1a or Brg1 at c-Myc binding sites, as measured by read counts (see Methods), was more robust than in non-overlapping regions.

The functional effects of the interaction between cBAF and c-Myc was examined. In activated CD8⁺ T cells deficient in Arid1a, accessible chromatin was reduced in ATAC-seq analysis, including accessible chromatin in gene elements associated with T_{EFF} function. In these Arid1a-deficient cells, chromatin binding of Arid1a and Brg1 was dramatically reduced, as expected. While the overall c-Myc binding in Arid1a-deficient activated CD8⁺ T cells was only modestly reduced, the reduced binding sites included gene elements critically involved in T_{EFF} differentiation and function, including Granzyme B, IL2Ra, and T-bet, to which c-Myc, Brg1, and Arid1a co-bind. Consistent with these observations, RNA-seq and gene set enrichment analyses of Arid1a-deficient first division CD8⁺ T cells revealed a reduction of c-Myc as well as TORC1 target genes. Conversely, in activated CD8⁺ T cells in which c-Myc had been acutely ablated (see Methods), the binding of Arid1a and Brg1 to chromatin was reduced, as revealed by the CUT&RUN assay. This reduced cBAF binding in the absence of c-Myc was associated with reduced chromatin accessibility assessed by ATAC-seq. Such impairment in cBAF binding was unlikely due to reduced expression of cBAF components in Myc-deficient cells, as transcriptome and immunoblot analyses revealed largely comparable expression of various cBAF components in WT and c-Myc-deficient CD8⁺ T cells. Instead, these results indicate that c-Myc likely facilitates stable CBAF binding and/or function at overlapping chromatin-binding sites in activated CD8⁺ T cells.

Previously, it had been shown that a partial reduction of c-Myc levels in Myc^{+/-} CD8⁺ T cells promotes the ability of CD8^{hi} first-division cells to contribute to T cell recall responses (Verbist et al. (2016) Nature 532:389-393).

Therefore, asymmetry of CBAF components and accessible chromatin was examined in the first division of WT and Myc^{+/-} CD8⁺ T cells. This analysis indicated that heterozygous loss of c-Myc significantly reduced the asymmetric distribution of accessible chromatin. Moreover, quantification of the asymmetric index (difference in fluorescence intensity/total), a parameter by which a higher value indicates a more pronounced asymmetric distribution of the target of interest (Verbist et al. (2016) Nature 532:389-393), also showed that heterozygous loss of c-Myc reduced the asymmetric distribution of accessible chromatin and Brg1, as well as a trended reduction for asymmetric distribution of Arid1a. These results, together with those above, indicate that c-Myc stabilizes and/or supports the chromatin-remodeling function of cBAF in activated CD8⁺ T cells, with such cooperation important for the ability of c-Myc to promote TE and suppress MP cell fate trajectories. To demonstrate this, c-Myc expression was enforced in activated CD8⁺ T cells. It was observed that ectopic c-Myc expression promoted expression of TE-associated markers (CX3CR1 and KLRG1) and reduced expression of memory-associated markers (CD27 and CXCR3), an effect that was essentially eliminated when the cBAF component Smarcd2 was ablated. These results collectively indicate that CBAF and c-Myc function cooperatively to promote T cell differentiation toward the TE cell fate.

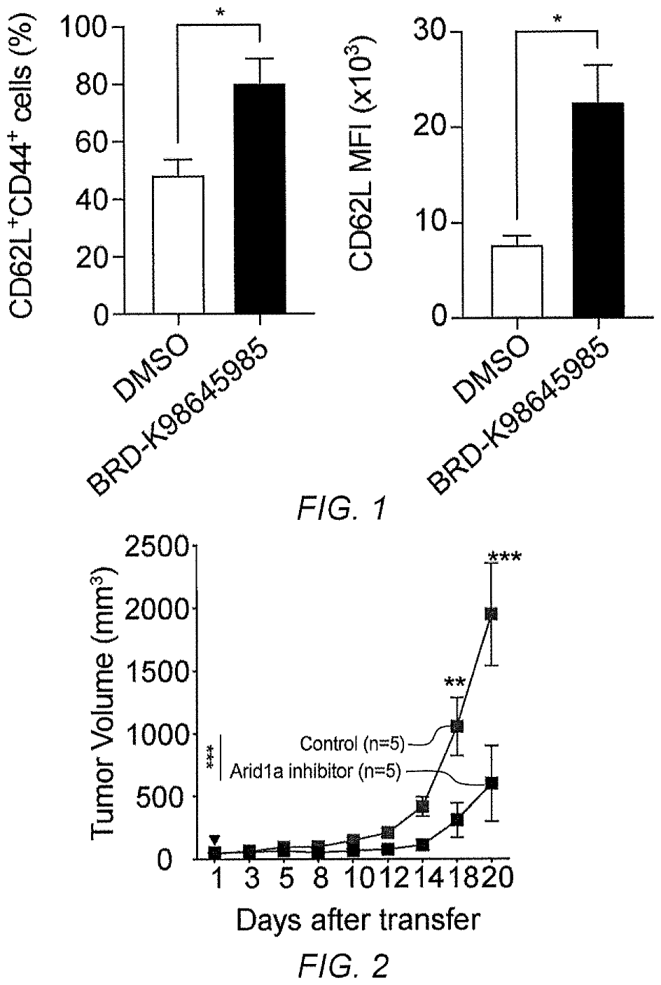

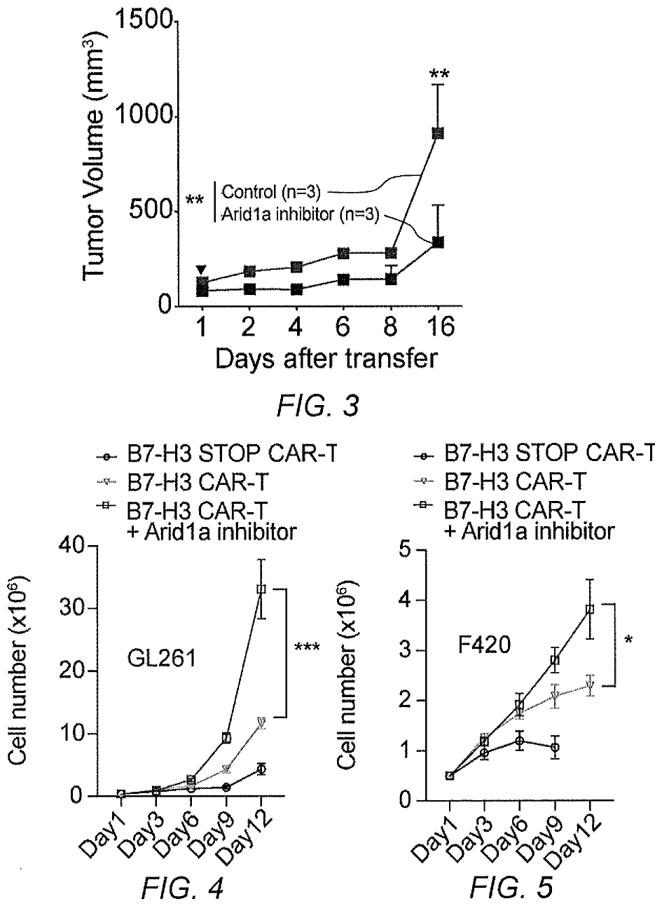

Effective adoptive T cell therapy for cancer is critically dependent upon the use of T cells with long-term memory potential (Lugli et al. (2020) Trends Immunol. 41:17-28; Gattinoni et al. (2012) Nat. Rev. Cancer 12:671-684; Klebanoff et al. (2005) Proc. Natl. Acad. Sci. USA 102:9571-9576; Hurton et al. (2016) Proc. Natl. Acad. Sci. USA 113: E7788-E7797). Therefore, it was determined whether manipulation of CBAF can improve anti-tumor therapy. Accordingly, OT-I cells were transduced with sgRNA for Smarcd2 or control sgRNA, and the transduced cells were transferred into animals that were subsequently infected with Lm-Ova. Animals were then inoculated with B16F10 murine melanoma cells expressing Ova (B16-Ova) at memory phase (day >30 post-infection). Improved control of tumor growth and enhanced survival by the Smarcd2-ablated CD8⁺ T cells were observed. The studies on c-Myc function in T cells indicated that c-Myc acts early in T cell activation to promote cell fate trajectories (Verbist et al. (2016) Nature 532:389-393). Therefore, if cooperation between cBAF and c-Myc similarly acts early, transient inhibition of cBAF may be sufficient to promote subsequent anti-cancer immunity. To demonstrate this, an inhibitor of Arid1a, BRD-K98645985 (Marian et al. (2018) Cell. Chem. Biol. 25:1443-1455; Chory et al. (2020) ACS Chem. Biol. 15:1685-1696), was used. OT-I cells were activated for 48 hours in the presence of the inhibitor (1 μM), and then the drug was removed and the cells were cultured for an additional 2 days in the presence of interleukin-2 (IL-2). As observed with Arid1a ablation, transient treatment of activated OT-I cells with an Arid1a inhibitor promoted the generation of cells with the TCM marker CD62L (FIG. 1). The OT-I T cells were then transferred into animals bearing palpable B16-Ova or MC38-Ova tumors. Transient treatment with the Arid1a inhibitor improved the ability of OT-I cells to control B16-Ova (FIG. 2) and MC38-Ova (FIG. 3) tumor growth in vivo. Therefore, transient inhibition of cBAF function during the activation of CD8⁺ T cells is effective in promoting their anti-tumor function.

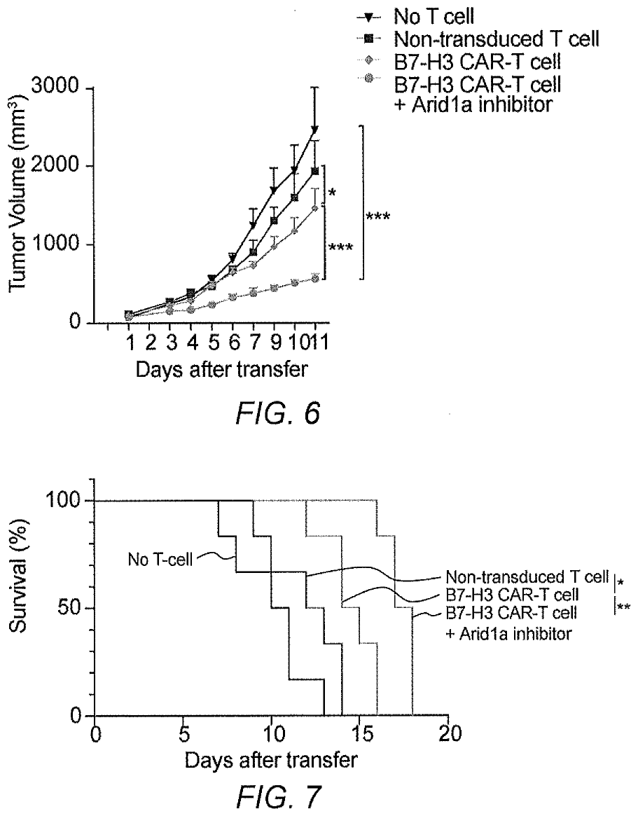

Currently the most widely used adoptive T cell therapy employs the use of CARs. However, the use of CAR-T cells for the treatment of solid tumors remains challenging (Lim & June whether CAR-T cell (2017) Cell 168:724-740). To examine function is enhanced by acute Arid1a inhibition, a CAR targeting murine B7-H3 (Haydar et al. (2021) Neuro-Oncol. 23:999-1011) was used in two B7-H3-positive cell lines, murine osteosarcoma F420 (Zhao et al. (2015) Oncogene 34:5069-5079; Kenneth et al. (2021) Mol. Ther. 29:326-326) and glioma GL261 (Haydar et al. (2021) Neuro-Oncol. 23:999-1011). Murine T cells were activated for 2 days in the presence or absence of the Arid1a inhibitor, followed by retroviral transduction of the B7-H3 CAR to generate CAR-T cells. After culturing for an additional 6 days, the T cells transiently treated with the inhibitor generated CAR-T cells characterized by high CD62L expression, while no difference in CAR expression was observed compared with vehicle-treated controls. Repeated stimulation with either of two B7-H3-expressing tumor lines resulted in a greater expansion of the CAR-T cells derived from T cells transiently treated with the Arid1a inhibitor (FIG. 4 and FIG. 5). The ability of B7-H3 CAR-T cells to control the growth of F420 cells was then assessed in vivo. While the CAR-T cells generated from activated T cells showed a modest ability to control tumor growth, those that had been transiently treated with the Arid1a inhibitor displayed markedly enhanced tumor control (FIG. 6) and survival (FIG. 7). Analysis of tumor infiltrating lymphocytes at day 9 post-transfer showed that the CAR-T cells generated from inhibitor-treated cells were in higher numbers than those produced from vehicle-treated CAR-T cells and comprised a higher proportion of CD62L⁺CD44⁺ cells. These results indicate that Arid1a inhibition enables better anti-tumor activity of CAR-T cells.

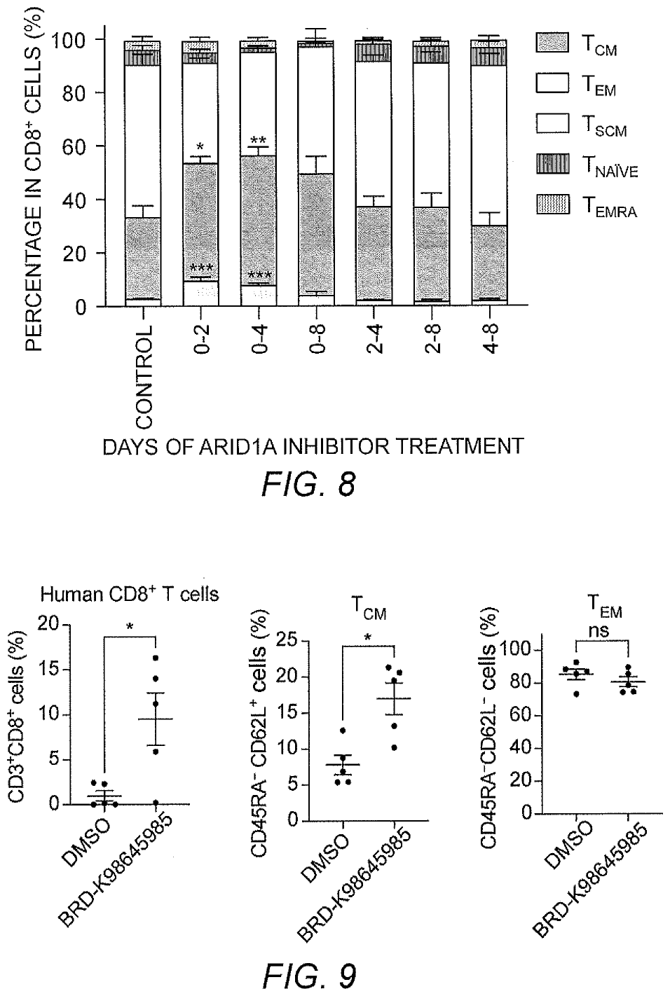

The effect of the Arid1a inhibitor on the differentiation of activated, human CD8⁺ T cells was also analyzed. Human naïve CD8⁺ T cells from healthy donors were enriched and stimulated with anti-CD3/CD28, following by culturing in IL-15 or IL-2, two cytokines that respectively promote in vitro T_{MEM}-like and T_{EFF}-like cell formation (Buck et al. (2016) Cell 166:63-76; Mitchell et al. (2010) J. Immunol. 184:6719-6730). Arid1a inhibitor was added during different periods of culture. It was found that treatment during the first 48 hours was necessary and sufficient to promote the generation of cells with markers of T_{CM} (CD45RA-CCR7+) and T memory stem cells (T_{SCM}, CD45RA⁺CCR7⁺CD27⁺CD95⁺; Lugli et al. (2020) Trends Immunol. 41:17-28) and reduced numbers of cells with markers of effector memory T cells (T_{EM}, CD45RA⁺CCR7⁻) (FIG. 8). Human CD8⁺ T cells were then activated with anti-CD3/CD28, with or without transient treatment with the Arid1a inhibitor and the cells were transferred to immunocompromised Nod-Scid-common gamma chain-deficient (NSG) mice. After 30 days, T cells were recovered and analyzed. It was found that transient treatment with the Arid1a inhibitor endowed the activated T cells with enhanced persistence in the animals, and a greater propensity to express markers of TCM cells (FIG. 9). These data therefore indicate that transient inhibition of cBAF function during T cell activation improves CAR-T therapy for human cancers.

Components of the chromatin remodeling cBAF complex are frequently mutated in human cancers (Centore et al. (2020) Trends Genet. 36:936-950; Mittal & Roberts (2020) Nat. Rev. Clin. Oncol. 17:435-448; Lu & Allis (2017) Nat. Genet. 49:178-179; Biegel et al. (2014) Am. J. Med. Genet. C Semin. Med. Genet. 166C: 350-366). In such settings, re-establishment of BAF function has been found to suppress expression of c-Myc (Weissmiller et al. (2019) Nat. Commun. 10:2014; Wang et al. (2019) Gut 68:1259-1270). In contrast, it has now been found that in activated CD8⁺ T cells, c-Myc appears to promote or stabilize the binding of cBAF to the chromatin and to cooperate with cBAF in shaping chromatin accessibility and gene expression programs. Further, this function of c-Myc appears to be required for its ability to propel activated CD8⁺ T cells toward a TE cell fate and away from a MP trajectory. Moreover, in the context of cancers with mutations in the cBAF complex, CBAF has been proposed to regulate “stemness” of cancer cells (Lu & Allis (2017) Nat. Genet. 49:178-179; Wang et al. (2011) J. Clin. Invest. 121:3834-3845). The finding that deletion or inhibition of CBAF components promotes T_{MEM} generation in the physiological immune response indicates that CBAF inhibits stem-like attributes in activated T cells. Indeed, ablation of the cBAF component, Smarcb1, in mice leads to fully penetrant CD8⁺ leukemia expressing a signature of T_{MEM} cells (Wang et al. (2011) J. Clin. Invest. 121:3834-3845). Collectively, these results point to both shared and context-specific mechanisms of the cBAF complex in various processes in vivo.

The fate of activated T cells is determined by many factors, including the strength of T cell receptor engagement, cytokines produced by antigen-presenting cells or in the local environment, metabolite, and nutrient availability, among others (Chapman et al. (2020) Nat. Rev. Immunol. 20:55-70; Jameson & Masopust (2018) Immunity 48:214-226; Chang et al. (2014) Nat. Immunol. 15:1104-1115; Charnley et al. (2019) J. Cell Sci. 133:432; Madden & Rathmell (2021) Cancer Discov. 11:1636-1643). Emerging evidence also reveals the importance of epigenetic programs in establishing lineage fate and stability (Henning et al. (2018) Nat. Rev. Immunol. 18:340-356; Bagert et al. (2021) Nat. Chem. Biol. 17:403-411; Wan et al. (2020) Nature 577:121-126), but the key epigenetic mechanisms, especially those that negatively control the memory cell fate trajectory, are elusive. Here, more than 300 epigenetic factors were screened using an unbiased in vivo pooled CRISPR screening strategy and it was found that cBAF complex was the most enriched epigenetic pathway that negatively regulated T_{MEM} formation and function. Extensive reprograming of the epigenetic program and chromatin state was found to occur during the first cell division, including the asymmetric sorting of the cBAF complex into two daughter cells. These findings provide key mechanistic insights into earlier observations that gene expression signatures of TE and MP can be detected as early as the first division of activated CD8⁺ T cells (Kakaradov et al. (2017) Nat. Immunol. 18:422-432; Chang et al. (2007) Science 315:1687-1691). Consistent with this early reprogramming, it was discovered that transient cBAF inhibition for the initial 2 days of TCR stimulation was sufficient to induce durable T_{MEM} responses in vivo, supporting a model of epigenetic inheritance. Therefore, the chromatin remodeling machinery is asymmetrically sorted at the first cell division, and even after the cessation of the primary stimulation, the epigenetic effects can engage a cell fate trajectory for durable responses.

What is claimed is:

1. A method for preparing T cells for adoptive T cell therapy comprising contacting a population of activated T cells with an AT-rich interaction domain 1A (Arid1a) inhibitor thereby preparing the T cells for adoptive T cell therapy.

2. The method of claim 1, wherein the T cells are CD8⁺ T cells.

3. The method of claim 1, wherein the population of activated T cells is contacted with the Arid1a inhibitor during the first 48 hours after activation.

4. The method of claim 3, wherein activation is via stimulation of CD3, CD28, or a combination thereof.

5. The method of claim 1, further comprises expanding the activated population of T cells with one or more cytokines.

6. The method of claim 1, further comprising introducing into said population of T cells an exogenous nucleic acid molecule thereby producing a population of engineered T cells.

7. The method of claim 6, wherein the exogenous nucleic acid molecule encodes an antigen recognizing receptor, an ortho-receptor, an immunomodulatory cytokine, a chemokine receptor, a dominant-negative receptor, or a transcription factor for preventing exhaustion.

8. The method of claim 7, wherein the antigen is a tumor antigen, a self-antigen, or a pathogen antigen.

9. The method of claim 7, wherein said antigen recognizing receptor is a T cell receptor (TCR) or a chimeric antigen receptor (CAR).

10. A population of T cells prepared by the method of claim 1.

11. A pharmaceutical composition comprising the population of T cells of claim 10 in admixture with a pharmaceutically acceptable carrier or vehicle.

12. A method for adoptive T cell therapy comprising administering to a subject in need of adoptive T cell therapy the pharmaceutical composition of claim 11.

13. A population of engineered T cells prepared by the method of claim 6.

14. A pharmaceutical composition comprising the population of engineered T cells of claim 13 in admixture with a pharmaceutically acceptable carrier or vehicle.

15. A method of treating cancer comprising administering to a subject in need of treatment the pharmaceutical composition of claim 14.

16. A kit for preparing T cells for adoptive T cell therapy comprising
(a) an AT-rich interaction domain 1A (Arid1a) inhibitor, and
(b) a CD3 agonist.

17. The kit of claim 16, further comprising (c) one or more costimulatory ligands.

18. The kit of claim 16, further comprising (c) one or more cytokines.
